(19) United States
(12) Patent Application Publication
(10) Pub. No.: US 2007/0219174 A1
(43) Pub. Date: Sep. 20, 2007

(54) METHODS OF TREATING CANCER USING HYPOFRACTIONATED RADIATION AND TEXAPHYRINS

(75) Inventors: Richard Miller, Portola Valley, CA (US); Markus Renschler, Redwood City, CA (US)

Correspondence Address: WILSON SONSINI GOODRICH & ROSATI, 650 PAGE MILL ROAD, PALO ALTO, CA, 94304-1050, US

(73) Assignee: Pharmacyclics, Inc., Sunnyvale, CA (US)

(21) Appl. No.: 11/276,827

(22) Filed: Mar. 15, 2006

(51) Int. Cl. A61K 31/555; A61N 5/00

(52) U.S. Cl. 514/185; 600/1

(57) ABSTRACT

Described herein are methods of treating cancer by administering a therapeutically effective amount of at least one texaphyrin metal complex or a pharmaceutically acceptable derivative, and performing stereotactic radiosurgery to the patient. In some embodiments, at least one texaphyrin metal complex or a pharmaceutically acceptable derivative is administered while the patient is undergoing a radiation therapy. Also described herein are methods for detecting various cancerous tumors, lesions, or metastases in a patient by administering an effective amount of at least one texaphyrin metal complex or a pharmaceutically acceptable derivative before a patient undergoes stereotactic radiosurgery. Also described herein are methods for visualizing tumors not otherwise visualized; methods for detecting the margins of a tumor; and methods for improving the targeting of the SRS radiation.

### BACKGROUND OF THE INVENTION

Cancer is a serious threat to modern society. Worldwide, more than 10 million people are diagnosed with cancer every year and it is estimated that this number will grow to 15 million new cases every year by 2020. Approximately half of cancer patients in the U.S. receive radiation therapy as part of initial disease management. Radiation therapy is often used to treat, for example only, but not limited to, brain metastases. Brain metastases are known to be a secondary manifestation of some forms of systemic cancer, including breast cancer, lung cancer, pancreatic cancer, melanoma, kidney cancer, and prostrate cancer. The majority of patients with brain metastases have neurological and neurocognitive impairments that prevent them from functioning independently.

Because normal tissue cannot tolerate extremely high doses of radiation, tumor-selective agents that enhance the ability of radiation to effect tumor growth have been developed to facilitate administration of lower doses of radiation. Thus, tumor selective radiation enhancers facilitate improved tumor reduction in response to radiation therapy, decrease radiation toxicity, and spare normal tissues.

Texaphyrin-metal complexes, such as Gadolinium Texaphyrin (Gd-Tex) and Motexafirin Gadolinium (MGd), have been shown to selectively localize in tumors and can be used as radiation enhancers. Because of their paramagnetic property, texaphyrin-metal complexes have been used to detect tumor localization with magnetic resonance imaging (MRI). In addition, by generating reactive oxygen species intracellularly, texaphyrin-metal complexes such as MGd can also lower the apoptotic threshold of cells to radiation and chemotherapy.

### SUMMARY OF THE INVENTION

Described herein are methods of treating cancer by administering at least one dose of an imageable texaphyrin metal complex or a pharmaceutically acceptable derivative, and providing a least one dose of at least 10 Gy to at least one, lesion in the patient. In further embodiments, the imageable texaphyrin metal complex is imageable by at least one method selected from: X-ray imaging, K-edge imaging, computerized tomography, magnetic resonance imaging, contrast-enhanced magnetic resonance imaging, positron emission tomography, optical imaging, fluorescence imaging, or a combination there of. In further embodiments, the imageable texaphyrin metal complex is a texaphyrin gadolinium complex. In further embodiments, the texaphyrin gadolinium complex is motexafin gadolinium. In further embodiments, the imageable texaphyrin metal complex preferentially localizes to at least one lesion, and in further embodiments localizes intracellularly within cancer cells.

In any such method described herein are methods further comprising the step of providing hyperfractionated radiation therapy on the patient. In a further or alternative embodiment, the hyperfractionated radiation therapy is whole brain radiation therapy (WBRT), wherein the WBRT in administered over 6-50 days. In further or alternative embodiments the hyperfractionated radiation therapy is stereotactic radiosurgery (SRS), wherein the SRS is administered (for example) over 2 days, in 3 days, in 4 days, in 5 days, in 6 days, in 7 days. In any such method described herein are methods further comprising the step of providing hypofractionated radiation therapy on the patient. In a further or alternative embodiment, the hypofractionated radiation therapy is whole brain radiation therapy (WBRT), wherein the WBRT in administered over 1-5 days. In further or alternative embodiments the hypofractionated radiation therapy is stereotactic radiosurgery (SRS) wherein the SRS is administered (for example) over 1 day.

In further or alternative embodiments, the patient is also administered at least one dose an imageable non-texaphyrin metal complex, including a non-texaphyrin gadolinium metal complex. In further embodiments, the imageable non-texaphyrin metal complex preferentially localizes to at least one lesion, and in further embodiments localizes extracellularly in the lesion. In further embodiments, the non-texaphyrin gadolinium metal complex is a gadolinium contrast agent.

In further embodiments, the step of providing at least one dose of at least 10 Gy to at least one lesion is stereotactic radiosurgery (“SRS”). In some embodiment, the patient receives at least 15 Gy to at least one lesion; at least 20 Gy to at least one lesion. In further embodiments, the lesion is selected from brain cancer, lung cancer, breast cancer, prostate cancer, colon cancer, stomach cancer, and liver cancer. In further embodiments, the lesion is metastatic cancer.

In some embodiments, the patient receives the imageable texaphyrin-metal complex at least about 4 hours prior to receiving stereotactic radiosurgery; at least about 3 hours prior to receiving stereotactic radiosurgery; at least about 2 hours prior to receiving stereotactic radiosurgery In some embodiments, the patient has received at least 5 doses of the texaphyrin metal complex in the three week period prior to SRS; at least 7 doses of the texaphyrin metal complex in the three week period prior to SRS; at least 10 doses of the texaphyrin metal complex in the three week period prior to SRS. In further embodiments, the multiple doses of the texaphyrin metal complex are provided to the patient every third day; every other day; or every fourth day. In further or alternative embodiments, at least one dose of the texaphyrin metal complex is provided within 1 week after the patient has received SRS; within 2 weeks after the patient has received SRS; within 3 weeks after the patient has received SRS; or within 4 weeks after the patient has received SRS.

In some embodiments, the patient has further received at least one dose of a non-texaphyrin gadolinium complex at least once in the three weeks prior to SRS. In some embodiment, the patient has received radiation therapy WBRT at least once in the three week period prior to SRS. In some embodiments, at least one texaphyrin-metal complex is MGd. In some embodiments, the texaphyrin-metal complex is MGd. In some embodiments, the cancer is brain metastases and the patient exhibits 1 to 4 brain metastases from a solid tumor. In some embodiments, the improved method provides increased image brightness of at least one lesion. In some embodiments, the improved method provides increased image sensitivity of at least one lesion. In some embodiments the improved method provides increased image accuracy for at least one lesion. In some embodiments, the improved method detects at least one additional lesion. In some embodiments, the improved method provides increased lesion identification. In some embodiments, the improved method provides increased lesion location. In some embodiments, the improved method provides increased lesion perimeter location.

In some embodiments, at least one texaphyrin metal complex or a pharmaceutically acceptable derivative is administered while the patient is undergoing a radiation therapy. Also described herein are methods for detecting various cancerous tumors, lesions, or metastases in a patient by administering an effective amount of at least one texaphyrin metal complex or a pharmaceutically acceptable derivative before a patient undergoes stereotactic radiosurgery.

In one aspect are methods of treating cancer in a patient, comprising:
(a) administering at least 5 mg/kg of a high-purity texaphyrin-metal complex or a pharmaceutically acceptable derivative to the patient; and
(b) providing, within at least 24 h after receiving a dose of a texaphyrin-metal complex, at least 10 Gy of radiation to at least one lesion in the patient.

In a further or alternative embodiment, the high-purity texaphyrin-metal complexes is Motexafin Gadolinium (MGd). In a further or alternative embodiment, the patient is further administered at least one dose of a non-texaphyrin gadolinium complex. In a further or alternative embodiment, the MGd is administered at least 4 hours prior to receiving at least 10 Gy to at least one lesion. In a further or alternative embodiment, the patient has been administered multiple doses of MGd in the three weeks prior to receiving at least 10 Gy to at least one lesion. In a further or alternative embodiment, at least five doses of MGd have been administered in the three weeks prior to receiving at least 10 Gy to at least one lesion. In a further or alternative embodiment are methods further comprising the step of providing hyperfractionated radiation therapy on the patient. In a further or alternative embodiment, the hyperfractionated radiation therapy is whole brain radiation therapy (WBRT), wherein the WBRT in administered over 6-50 days. In further or alternative embodiments the hyperfractionated radiation therapy is stereotactic radiosurgery (SRS), wherein the SRS is administered (for example) over 2 days, in 3 days, in 4 days, in 5 days, in 6 days, in 7 days. In a further or alternative embodiment are methods further comprising the step of providing hypofractionated radiation therapy on the patient. In a further or alternative embodiment, the hypofractionated radiation therapy is whole brain radiation therapy (WBRT), wherein the WBRT in administered over 1-5 days. In further or alternative embodiments the hypofractionated radiation therapy is stereotactic radiosurgery (SRS), wherein the SRS is administered (for example) over 1 day. In a further or alternative embodiment, the at least 10 Gy of radiation is provided by stereotactic radiosurgery. In a further or alternative embodiment, the high-purity texaphyrin-metal complex or a pharmaceutically acceptable derivative is administered prior to each time the patient receives hyperfractionated radiation therapy. In a further or alternative embodiment, the patient receives MGd during the second and third week of treatment of WBRT. In a further or alternative embodiment, the patient receives a total of about 30 to about 50 Gy of radiation during WBRT. In further or alternative embodiment, the patient receives a total of about 35 to about 40 Gy of radiation during WBRT. In a further or alternative embodiment, the patient receives at least about 15 Gy of radiation to at least one lesion. In a further or alternative embodiment, about ten doses of MGd in an amount of up to about 5 mg/kg is administered daily during the second and third week of treatment of WBRT. In a further or alternative embodiment, the cancer's size is reduced by at least about 50%. In a further or alternative embodiment, radiological progression in the patient is reduced by at least about 50%. In a further or alternative embodiment, the patient exhibits about 1 to 4 brain metastases from a solid tumor. In a further or alternative embodiment, neurological progression in the patient is reduced by at least about 50%.

Another aspect described herein is an improved method for treating cancer in a patient undergoing radiation therapy, wherein the improvement is administering an effective amount of a high-purity texaphyrin-metal complex or a pharmaceutically acceptable derivative before the patient receives stereotactic radiosurgery. In a further or alternative embodiment, the radiation therapy further comprises WBRT. In a further or alternative embodiment, the high-purity texaphyrin-metal complex is MGd. In a further or alternative embodiment, the patient is further administered at least one dose of a non-texaphyrin gadolinium complex. In a further or alternative embodiment, the cancer is brain metastases and the patient exhibits 1 to 4 brain metastases from a solid tumor. In a further or alternative embodiment, the brain metastases' size is reduced by at least about 50%. In a further or alternative embodiment, neurological progression in the patient is reduced by at least about 50%. In a further or alternative embodiment, radiological progression in the patient is reduced by at least about 50%.

In another aspect are methods of treating metastases in a patient, comprising:
(a) administering an effective amount of motexafin gadolinium (MGd) to the patient; and
(b) performing stereotactic radiosurgery on the area of the patient's body comprising the metastases.

In a further or alternative embodiment, the MGd is administered prior to performing the stereotactic radiosurgery on the patient. In a further or alternative embodiment, further comprising the step of performing WBRT on the patient. In a further or alternative embodiment, the MGd is further administered prior to the patient undergoing WBRT. In a further or alternative embodiment the patient exhibits 1 to 4 metastases from a solid tumor. In a further or alternative embodiment, the brain metastases' size is reduced by at least about 50%. In a further or alternative embodiment, neurological progression in the patient is reduced by at least about 50%. In a further or alternative embodiment, radiological progression in the patient is reduced by at least about 50%.

In another aspect are methods of treating glioblastoma multiforme in a patient, comprising:
(a) administering an effective amount of motexafin gadolinium (MGd) to the patient; and
(b) performing stereotactic radiosurgery on the patient at afflicted areas.

In a further or alternative embodiment, the MGd is administered prior to performing the stereotactic radiosurgery on the patient. In a further or alternative embodiment, further comprising the step of performing external beam radiation therapy on the patient. In a further or alternative embodiment, the MGd is administered prior lo the patient undergoing external beam radiation therapy. In a further or alternative embodiment, the brain metastases' size is reduced by at least about 50%. In a further or alternative embodiment, neurological progression in the patient is reduced by at least about 50%. In a further or alternative embodiment, radiological progression in the patient is reduced by at least about 50%.

In another aspect are improved methods for defining the treatment field for brain metastases in a patient, wherein the improvement is administering an effective amount of at least one texaphyrin-metal complex or a pharmaceutically acceptable derivative before the patient receives stereotactic radiosurgery. In a further or alternative embodiment, at least one texaphyrin-metal complex is MGd. In a further or alternative embodiment, the patient is further administered at least one dose of a non-texaphyrin gadolinium complex.

A method for detecting at least one cancerous tumor, lesion, or metastases in a patient's brain, comprising:
(a) administering an effective amount of at least one texaphyrin-metal complex or a pharmaceutically acceptable derivative to the patient before undergoing stereotactic radio surgery;
(b) screening the patient's brain; and
(c) detecting the presence of at least one texaphyrin-metal complex or a pharmaceutically acceptable derivative, thereby detecting the tumor, lesion, or metastases.

In a further or alternative embodiment, at least one texaphyrin-metal complex is MGd. a further or alternative embodiment, the patient is further administered at least one dose of a non-texaphyrin gadolinium complex.

A method for identifying tumor lesions or metastases in a patient, comprising:
(a) administering an effective amount of at least one texaphyrin-metal complex or a pharmaceutically acceptable derivative to the patient before undergoing stereotactic radio surgery;
(b) screening the area of the patient's body comprising the lesions or metastases; and
(c) detecting the presence of at least one texaphyrin-metal complex or a pharmaceutically acceptable derivative, whereby the location and volume of tumor lesions are identified.

In a further or alternative embodiment, at least one texaphyrin-metal complex is MGd. a further or alternative embodiment, the patient is further administered at least one dose of a non-texaphyrin gadolinium complex.

An improved method for identifying tumor lesions or metastases in a patient prior to stereotactic radiosurgery, wherein the improvement is administering an effective amount of at least one texaphyrin-metal complex or a pharmaceutically acceptable derivative to the patient. In a further or alternative embodiment, at least one texaphyrin-metal complex is MGd. In a further or alternative embodiment, the patient is further administered at least one dose of a non-texaphyrin gadolinium complex.

One aspect described herein relates to a method of treating cancer in a patient, comprising: (a) administering a therapeutically effective amount of at least one texaphyrin-metal complex or a pharmaceutically acceptable derivative to the patient; and (b) performing stereotactic radiosurgery on the patient. In some embodiments, at least one of the texaphyrin-metal complexes is Motexafin Gadolinium (MGd). In some embodiments, the texaphyrin-metal complex is MGd. In some embodiments, the MGd is administered prior to performing the stereotactic radiosurgery. In some embodiments, up to about 5 mg/kg of MGd is administered to the patient. In some embodiment, a total of about ten doses of MGd in an amount of up to about 5 mg/kg are administered to the patient.

In some embodiment, the method further comprises the step of performing radiation therapy on the patient. In some embodiment., the radiation therapy is whole brain radiation therapy (WBRT). In some embodiment, the patient receives multiple treatments of WBRT over a three week time period. In some embodiment, at least one texaphyrin-metal complex or a pharmaceutically acceptable derivative is administered prior to the patient undergoing radiation therapy. In some embodiment, the patient receives MGd during the second and third week of treatment of WBRT. In one embodiment, the patient receives a total of about 30 to about 50 Gy of whole brain radiation therapy. In another embodiment, the patient receives a total of about 35 to about 40 Gy of whole radiation therapy.

When the patient receives doses of WBRT over about a three week time period, in some embodiments, doses of MGd in an amount of up to about 5 mg/kg is administered during the second and third week of treatment of WBRT. When the method of treating cancer is employed, the cancer's size is reduced by at least about 50% In some embodiments, radiological progression in the patient is reduced by at least about 50%. In some embodiments, the patient exhibits about 1 to 4 brain metastases from a solid tumor. In some embodiments, neurological progression in the patient is reduced by at least about 50%.

Another aspect relates to an improved method for treating cancer in a patient undergoing radiation therapy, wherein the improvement is administering an effective amount of at least one texaphyrin-metal complex or a pharmaceutically acceptable derivative before the patient receives stereotactic radiosurgery. In some embodiments, the patient receives the texaphyrin-metal complex at least about 4 hours prior to receiving stereotactic radiosurgery; at least about 3 hours prior to receiving stereotactic radiosurgery; at least about 2 hours prior to receiving stereotactic radiosurgery. In some embodiment, the radiation therapy includes WBRT. In some embodiments, at least one texaphyrin-metal complex is MGd. In some embodiments, the texaphyrin-metal complex is MGd In some embodiments, the cancer is brain metastases and the patient exhibits 1 to 4 brain metastases from a solid tumor. In some embodiments, the brain metastases' size is reduced by at least about 50%. In some embodiments, neurological progression in the patient is reduced by at least about 50%. In some embodiments, radiological progression in the patient is reduced by at least about 50%.

Another aspect relates to a method of treating metastases in a patient, comprising: (a) administering an effective amount of motexafin gadolinium (MGd) to the patient; and (b) performing stereotactic radiosurgery on the area of the patient's body comprising the metastases. In some embodiments, the MGd is administered prior to performing the stereotactic radiosurgery on the patient. In some embodiments, the method further comprises the step of performing WBRT on the patient. When the method further comprises the step of performing WBRT on the patient, the MGd can be administered while the patient is undergoing WBRT.

When a method of treating metastases in a patient is employed as described herein, in some embodiments, the patient exhibits 1 to 4 metastases from a solid tumor. In some embodiments the brain metastases' size is reduced by at least about 50%. In some embodiments, neurological progression in the patient is reduced by at least about 50%. In some embodiments, radiological progression in the patient is reduced by at least about 50%.

Another aspect relates to a method of treating glioblastoma multiforme in a patient, comprising: (a) administering an effective amount of motexafin gadolinium (MGd) to the patient; and (b) performing stereotactic radiosurgery on the patient at afflicted areas. In some embodiments the MGd is administered prior to performing the stereotactic radiosurgery on the patient. In some embodiments, the method further comprises the step of performing WBRT on the patient. When the method further comprises the step of performing WBRT on the patient, in some embodiments the MGd is administered prior to and/or after the patient undergoes WBRT. When a method of treating glioblastoma multiforme in a patient is employed as described herein, in some embodiments the brain metastases' size is reduced by at least about 50%. In some embodiments, neurological progression in the patient is reduced by at least about 50%. In some embodiments, radiological progression in the patient is reduced by at least about 50%.

Another aspect relates to an improved method for defining the image and treatment field for stereotactic radiosurgery, wherein the improvement is administering an effective amount of at least one texaphyrin-metal complex or a pharmaceutically acceptable derivative before the patient receives stereotactic radiosurgery. In some embodiments, the patient receives the texaphyrin-metal complex at least about 4 hours prior to receiving stereotactic radiosurgery; at least about 3 hours prior to receiving stereotactic radiosurgery; at least about 2 hours prior to receiving stereotactic radiosurgery. In some embodiments, the patient has received at least 5 doses of the texaphyrin metal complex in the three week period prior to SRS; at least 7 doses of the texaphyrin metal complex in the three week period prior to SRS; at least 10 doses of the texaphyrin metal complex in the three week period prior to SRS. In some embodiments, the patient has further received at least one dose of a non-texaphyrin gadolinium complex at least once in the three weeks prior to SRS. In some embodiment, the patient has received radiation therapy WBRT at least once in the three week period prior to SRS. In some embodiments, at least one texaphyrin-metal complex is MGd. In some embodiments, the texaphyrin-metal complex is MGd. In some embodiments, the cancer is brain metastases and the patient exhibits 1 to 4 brain metastases from a solid tumor. In some embodiments, the improved method provides increased image brightness of at least one lesion. In some embodiments, the improved method provides increased image sensitivity of at least one lesion. In some embodiments, the improved method provides increased image accuracy for at least one lesion. In some embodiments, the improved method detects at least one additional lesion. In some embodiments, the improved method provides increased lesion identification. In some embodiments, the improved method provides increased lesion location. In some embodiments, the improved method provides increased lesion perimeter location.

Another aspect relates to an improved method for detecting brain metastases in a patient wherein the improvement is administering an effective amount of at least one texaphyrin-metal complex or a pharmaceutically acceptable derivative before the patient receives stereotactic radiosurgery. In some embodiments, at least one texaphyrin-metal complex is MGd. In some embodiments, the texaphyrin-metal complex is MGd.

Another aspect relates to a method for detecting at least one cancerous tumor, lesion, can metastases in a patient's brain, comprising: (a) administering an effective amount of at least one texaphyrin-metal complex or a pharmaceutically acceptable derivative to the patient before undergoing stereotactic radiosurgery; (b) screening the patient's brain; and (c) detecting the presence of at least one texaphyrin-metal complex or a pharmaceutically acceptable derivative, thereby detecting the tumor, lesion, or metastases. In some embodiments, at least one texaphyrin-metal complex is MGd. In some embodiments, the texaphyrin-metal complex is MGd.

Another aspect relates to a method for identifying tumor lesions or metastases in a patient, comprising: (a) administering an effective amount of at least one texaphyrin-metal complex or a pharmaceutically acceptable derivative to the patient before undergoing stereotactic radiosurgery; (b) screening the area of the patient's body comprising the lesions or metastases; and (c) detecting the presence of at least one texaphyrin-metal complex or a pharmaceutically acceptable derivative, whereby the location and volume of tumor lesions are identified. In some embodiments at least one texaphyrin-metal complex is MGd. In some embodiments, the texaphyrin-metal complex is MGd.

Another aspect relates to an improved method for identifying tumor lesions or metastases in a patient prior to stereotactic radiosurgery, wherein the improvement is administering an effective amount of at least one texaphyrin-metal complex or a pharmaceutically acceptable derivative to the patient. In some embodiments, at least one texaphyrin-metal complex is MGd. In some embodiments, the texaphyrin-metal complex is MGd.

As used herein, the following terms have the given meaning unless otherwise specifically noted. It must be noted that, as used in the specification and the appended claims, the singular forms “a,” “an” and “the” include plural referents unless the context clearly dictates otherwise. Definition of standard chemistry terms may be found in reference works, including Cares and Sundberg, ADVANCED ORGANIC CHEMISTRY 3^{RD} ED. vols. A and B, Plenum Press, New York (1992). Unless otherwise indicated, conventional methods of mass spectroscopy, NMR, HPLC, protein chemistry, oncology, biochemistry, recombinant DNA techniques and pharmacology, within the skill of the art are employed.

The term “about” refers to an approximation of a specified quantity. When used in the context of a measured quantity, the word “about” includes amounts attributable to error, such an amounts which fall within a particular standard errors of mean, standards of deviation, and/or percent confidence limit. The word “about” may also include amounts resulting from an inherent error rate present in the particular method of detection, screening, or measurement being employed The term “about” includes amounts within at least 0.01%, 0.05%, 0.1%, 0.5%, 1.0%, 1.5%, 2.0%, 3.0%, 4.0%, 5.0%, 6.0%, 7.0%, 8.0%, 9.0%, or 10.0% of a specific quantity, as appropriate for the technique employed.

The term “during” refers to the occurrence of an act or event through the course or duration of a particular time period. In embodiments, the same or different events may occur simultaneously (e.g., concurrently with) or sequentially (e.g., before or after) within the specified time period.

The term “radiological progression” includes a change in radiological signs of a patient's tumor, metastases, lesions, and other malignant abnormality. Radiological progression includes the occurrence of new brain metastases, the development of leptomeningeal involvement the reappearance of old lesions, an increase in a lesion size (e.g., area, width, volume) or the sum of all lesion sizes (as determined, for example, by the product of the longest perpendicular diameters), and a change in shape or density of a lesion shape, a change in density of a lesion.

The term “neurological progression” includes a change (going from better to worse, i.e., worsening) in clinically detectable signs and symptoms controlled by the central nervous system (e.g., neuromuscular, central, peripheral, autonomic and the like). Assays and protocols for measuring neurological progression are well known to one of skill in the art, and is reported, for example, by Mehta et al., Journal of Clinical Oncology 20(16): 3445-3453 (2002), the disclosure of which is hereby incorporated by reference.

The term “memory function” includes the ability of a patient to retain information. Assays and protocols for measuring memory function are well known to one of skill in the art, and is reported, for example, by Mehta et al.

The term “executive function” includes the ability of a patient to recognize patterns and demonstrate abilities to plan and organize. Assays and protocols for measuring executive function are well known to one of skill in the art, and is reported, for example, by Mehta et al.

The term “statistically significant” means that an observation or an event is not attributed to random chance, p=0.05 or better.

The term “purified sample” or “purified composition” refers to a composition having a high degree of uniformity in the chemical structure (or molecular weight, excluding isotopic variations) of compounds having a particular generic chemical formula. That is, multiple compounds in a composition may be species of a particular generic chemical formula; however in a purified sample or purified composition, a high proportion of such compounds have the same chemical formula (or molecular weight, excluding isotopic variations). The value for the terms “high degree” or “high proportion” are provided specific values herein, for example, at least about 98.4%, at least about 98.7%, and the like. A purified sample or purified composition can include other chemical entities, including solvents, salts, reagents, pharmaceutical excipients, additional therapeutic agents, and the like. The defining aspect is that the composition has a high degree of uniformity in the chemical structure (or molecular weight, excluding isotopic variations) of compounds having a particular generic chemical formula (or molecular weight, excluding isotopic variations). The uniformity of a particular purified composition or purified sample can be determined by a variety of analytical methods, including by way of example only, chromatography and spectroscopic or spectrometric methods.

The terms “pharmaceutically effective amount”, “therapeutically effective amount” and “effective amount” as described herein, refers to a nontoxic but sufficient amount of the agent to provide the desired biological, therapeutic, and/or prophylactic result. The desired results include reduction and/or alleviation of the signs, symptoms, or causes of a disease, or any other desired alteration of a biological system. An effective amount in any individual case may be determined by one of ordinary skill in the art using routine experimentation.

The term “pharmaceutically acceptable” or “pharmacologically acceptable” as described herein, may mean a material which is not biologically or otherwise undesirable, i.e., the material may be administered to an individual without causing any undesirable biological effects or interacting in a deleterious manner with any of the components of the composition in which it is contained.

The term “patient” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals, such as rabbits, dogs, and cats; laboratory animals including rodents, such as rats, mice and guinea pigs, and the like. Examples of non-mammals include, but are not limited to, birds, fish and the like. In one embodiment, the mammal is a human

The term “treat”, “treating”, and its grammatical equivalents as described herein, refers to achieving, or attempting to achieve, a therapeutic benefit and/or a prophylactic benefit. By therapeutic benefit is meant eradication or amelioration, at least in part, of the underlying disorder being treated. For example, in a cancer patient, therapeutic benefit includes eradication or amelioration, at least in part, of the underlying cancer. Also, a therapeutic benefit includes the eradication or amelioration, at least in part, of one or more of the physiological symptoms associated with the underlying disorder such that an improvement is observed in the patient, notwithstanding the fact that the patient may still be afflicted with the underlying disorder. For prophylactic benefit a method disclosed herein may be performed on, or a composition disclosed herein administered t a patient at risk of developing cancer, or to a patient reporting one or more of the physiological symptoms of such conditions, even in the absence of a diagnosis of the condition.

The term “radiation therapy” as described herein, refers to exposing a patient to radiation, including without limitation electrons, x-rays, gamma rays, and neutrons. This type of therapy includes, without limitation, stereotactic therapy, whole brain therapy, external beam therapy, internal radiation therapy, implant radiation, brachytherapy, systemic radiation therapy, and other forms of radiation therapy well known to one of skill in the art.

The term “whole brain radiation therapy” (WBRT) refers to a form of radiation therapy delivered to the entire cranial content. The type of radiation, the size and number of radiation beams, the dose of radiation administered, and the duration of treatment depends on the particular tumor, metastases, or lesion being treated, and optimization of these factors is well known to one of skill in the art.

The term “external beam radiation therapy” refers to a form of radiation therapy wherein one or more beams of high-energy x-rays are delivered to a patient's tumor. The beam is generated outside the patient (such as by a linear accelerator) and is targeted at the tumor site without placement of radioactive sources within the patient's body. The type of radiation, the size and number of beams, the dose of radiation administered, and the duration of treatment depends on the particular tumor, metastases, or lesion being treated, and optimization of these factors is well known to one of skill in the art. External beam radiation therapy includes, but is not limited to, three-dimensional conformal radiation therapy (3D-CRT), conformal proton beam radiation therapy and intensity modulated radiation therapy (INIRT).

The term “stereotactic radiosurgery” refers delivery of highly focused beams of intense radiation to specific areas of the body. All forms of stereotactic radiosurgery are embraced including stereotactic radiosurgery performed with a gamma knife, linear accelerator, particle bean (proton) or cyclotron, instruments which are commercially available under the trade names Gamm Knife®, X-Knife®, SynergyS®, Trilogy®, CyberKnife®, Novalis®. Stereotactic radiosurgery is primarily used to treat tumors, metastases, lesions, and other malignant as well as non-malignant abnormalities. Stereotactic radiosurgery is a non-surgical, non-invasive procedure that exposes only a small afflicted area of the body, such as areas less than about 3.0, 1.0, 0.5, 0.25, 0.1, or 0.01 cm diameter, to beams of intense radiation in order to minimize exposure to normal areas and tissues the body. The type of radiation, the size and number of radiation beams, the dose of radiation administered, and the duration of treatment depends on the particular tumor, metastases, or lesion being treated, and optimization of these factors is well known to one of skill in the art.

Stereotactic radiosurgery is a precise form of radiation therapy used primarily to treat tumors, metastases, lesions, and other malignant as well as non-malignant abnormalities. Stereotactic radiosurgery is a non-surgical, non-invasive procedure that delivers focused beams of intense radiation to specific areas of the body. In this procedure, only afflicted areas of the body are exposed to the beams of intense radiation in order to minimize exposure to normal areas and tissues of the body. Stereotactic radiosurgery involves delivery of a single high-dose of radiation beams, or smaller, multiple doses of radiation beams converging in three dimensions to focus on a specific area of the body where the tumor or other abnormality resides. Stereotactic radiosurgery can be completed in a one-day session or, depending on the physician's recommendations, can be completed over a period of days or weeks. Stereotactic radiosurgery can be repeated on a daily basic if necessary.

In this procedure, a physician will first determine the location, size, shape, and volume of the tumor, metastases, lesion, or abnormality using conventional visualization techniques, such as magnetic resonance imaging (MRI), computed tomography (CT) scan, and/or a catheter angiogram. To facilitate targeted delivery of radiation beams, in some forms of SRS, the afflicted area of the body is immobilized and marked. Immobilization devices which provide means to mark particular areas of the body have been used to assist in targeting administration of radiation beams. See, for example, Bentel, G. C., Treatment Geometry, Patient Positioning and Immobilization in Radiation Oncology, McGraw-Hill Publishers, p. 1-10 (1999), Bentel, G. C., Treatment Accuracy and Precision, Patient Positioning and Immobilization in Radiation Oncology, McGraw-Hill Publishers, p. 11-22 (1999); Bentel, G. C., General Consideration of Positioning and Immobilization Patient Positioning and Immobilization in Radiation Oncology, McGraw-Hill Publishers, p. 23-37 (1999); and Bentel, G. C., Central Nervous System, Patient Positioning and Immobilization in Radiation Oncology, McGraw-Hill Publishers, p. 71-91 (1999). Head frames, which optionally have three-dimensional coordinates built in and can be attached to the skull with four screws, have also been used as guiding devices to ensure that radiation beams are focused exactly and only at positions where treatment is needed. Alternatively, some SRS systems use bony landmarks or beads in the ears and don't require immobilization of the patient.

Stereotactic radiosurgery is commonly used on the brain and is ideal for treating tumors that are lodged too deeply for surgical excision (i.e. inoperable tumors). Stereotactic radiosurgery can be used to treat many types of brain tumors, both benign or malignant and primary or metastatic. Additionally, stereotactic radiosurgery has been effective in treating patients with arteriovenous malformations (AVMs) i.e., a tangle of expanded blood vessels that disrupts normal blood flow in the brain and is the leading cause of stroke in young people. Furthermore, patients exhibiting acoustic neuromas have also been treated effectively with stereotactic radiosurgery.

Similar to conventional radiation treatment regimens, the intense X-rays employed a stereotactic radiosurgery do not actually remove the tumor, but instead, distorts the DNA of tumor cells. As a result, cancer cells lose their ability to reproduce. Following the treatment, benign tumors usually shrink over a period of 18 months to two years. Malignant and metastatic tumors may shrink more rapidly, even within a couple of months. When treated with radiosurgery, arteriovenous malformations begin to thicken and close off. All forms of stereotactic radiosurgery are embraced within the scope of the invention.

The term “chemotherapy” as described herein, refers to the administration of one or more anti-cancer drugs and/or other agents to a cancer patient by various methods, including intravenous, oral, intramuscular, intraperitoneal, intravesical, subcutaneous, transdermal, buccal, or inhalation or in the form of a suppository.

The term “parenteral administration” as used herein, refers to administration of at least one agent by means other than through the alimentary tract. Parenteral routes of administration involve injections into various compartments of the body such as but not limited to, intravenous, subcutaneous, intramuscular, intraperitoneal and the like.

The term “photodynamic therapy” as used herein, refers to a treatment that combines a light source and a photosensitizing agent (a drug that is activated by light).

The term “surgery” as used herein, refers to any therapeutic or diagnostic procedure that involves methodical action of the hand or of the hand with an instrument, on the body of a human or other mammal, to produce a curative, remedial, or diagnostic effect.

The term “metastases” refers to the spread of cancerous cells stemming from one part of the body to another. Metastases include formation of lesions and tumors, also referred to as “secondary tumors”, which comprise cells from the original (primary) tumor. When a certain type of cancer spreads to another part of the body, it generally does not change its type. For example, if a person with a lymphoma develops a tumor in the lung which is a metastasis from this lymphoma, the tumor growing in the lung has the same characteristics as the lymphoma, and does not represent a new lung cancer of the type which would develop if the cancer was to start in the lung. Metastases can occur through one or more various routes, including through the lymphatic system (in a process called “embolization”), bloodstream (e.g., through veins and/or arteries), by spreading through body spaces such as the bronchi or abdominal cavity, and/or through implantation or inoculation (e.g., through a spill of malignant cells from a needle or instrument during a biopsy or surgery). Some parts of the body are more vulnerable to becoming metastatic sites, such as the liver and lungs, than others body parts, such as the skin. Each type of cancer has its own pattern for metastases.

The term “brain metastases” refers to metastases, as defined above, occurring in any part of the brain. Brain metastases most often arise from lung or breast cancers. Other primary tumors that metastasize to the brain include melanoma, sarcomas, and tumors arising in the kidney or colon. In addition, primary cancers of unknown origin sometimes present with brain metastases

The term “glioblastoma multiforme” of “GBM” refers to a type of primary brain tumor. GBM is an anaplastic, highly cellular tumor with poorly differentiated, round, or pleomorphic cells, occasional multinucleated cells, nuclear atypia, and anaplasia. Variants of the tumor include gliosarcoma, multifocal GBM, or gliomatosis cerebri (in which the entire brain may be infiltrated with tumor cells). GBM seldomly metastasizes to the spinal cord or outside the nervous system. GBM is graded by their microscopic and histological appearance. Generally, grade I (pilocytic astrocytomas) and grade II (benign astrocytomas) tumors grow slowly over many years while grade IV (GBM) grows rapidly, invading and altering brain function.

### INCORPORATION BY REFERENCE

Unless stated otherwise, all publications and patent applications mentioned in this specification are herein incorporated by reference to the same extent as if each individual publication or patent application was specifically and individually indicated to be incorporated by reference.

### BRIEF DESCRIPTION OF THE DRAWINGS

FIG. 1A shows that MGd uptake enhances brain metastases not visible with standard Gd contrast.

FIG. 1B shows that MGd localization in brain metastases from NSCLC.

### DETAILED DESCRIPTION OF THE INVENTION

Texaphyrin Metal Complexes

Disclosed herein are methods using compositions comprising high-purity texaphyr metal complexes, wherein the identity of the substituents on the texaphyrin complex is at least about 98% identical on all texaphyrin complexes in a sample. Also disclosed herein are methods using pharmaceutical compositions comprising such high-purity texaphyrin metal complexes.

“Texaphyrin” means an aromatic pentadentate macrocyclic expanded porphyrins, and described as an aromatic benzannulene containing both 18π- and 22π-electron delocalization pathways. Texaphyrins and water-soluble texaphyrins, methods of preparation and various uses and the like have been described, for example, in U.S. Pat. Nos. 4,935,498, 5,162,509, 5,252,720, 5,256,399, 5,272,142, 5,292,414, 5,369,101, 5,432,171, 5,439,570, 5,451,576, 5,457,183, 5,475,104, 5,504,205, 5,525,325, 5,530,122, 5,559,207, 5,565,552, 5,567,687, 5,569,759, 5,580,543, 5,583,220, 5,587,371, 5,587,463, 5,591,422, 5,594,136, 5,595,726, 5,599,923, 5,599,928, 5,601,802, 5,607,924, 5,622,946, 5,714,328, 5,733,903, 5,744 302, 5,756,726, 5,763,172, 5,775,339, 5,776,925, 5,798,49 , 5,801,229, 5,808,059, 5,817,017, 5,837,866, 5,886,173, 5,888,997, 5,955,586, 5,969,111, 5,994,935, 6,022,526, 6,022,959, 6,069,140, 6,072,038 6,096,030, 6,207,660, 6,270,749, 6,375,930, 6,638,924, 6,657,058, 6,825,186, 6,919,327, in PCT publications WO 90/10633, 94/29316, 95/10307, 95/21845, 96/09315, 96/40253, 96/38461, 97/26915, 97/35617, 97/46262, 98/07733, 98/25648, 99/09411, 99/15236, 99/62551, 00/01413, 00/01414, 03/37888; 05/112759; and in pending U.S. patent application Ser. Nos. 10/160,205, 10/659,499, 10/310,592, 10/363,401, 10/362,964, 10/318, 659, 10/911,284, 11/241,549, 11/235,475, and 60/737,601, each of which are herein incorporated by reference in their entirety.

Exemplary compositions comprising high-purity texaphyrin metal complexes are described, for example, in U.S. patent application Ser. No. 11/235,475, the disclosure of which is hereby incorporated by reference.

The high-purity compositions of texaphyrin metal complexes employed herein comprise at least one texaphyrin metal complex according to Formula (I): 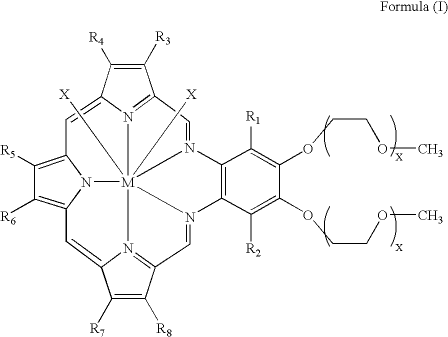
wherein: M is a trivalent metal cation selected from the group consisting of Gd⁺³ and Lu⁺³; each X is independently selected from the group consisting of OH⁻, AcO⁻, Cl⁻, Br⁻, I⁻, F⁻, H₂PO₄⁻, ClO⁻, ClO Cl₃⁻, ClO₄⁻, HCO₃⁻, HSO₄⁻, NO₃⁻, N₃⁻, CN⁻, SCN⁻, and OCN⁻; R₃, R4, R₅, R₆, R₇and R₈ are each independently H, OH, Cₙ H₍₂ₙ₊₁₎ O_{y} or OCₙ H₍₂ₙ₊₁₎ O_{y}; R₁, R₂ are independently H or C₁-C₆ alkyl; where at least one of R₃, R₄, R₅, R₆ , R₇ and R₈ is Cₙ H₍₂ₙ₊₁₎ O_{y} or OCₙ H₍₂ₙ₊₁₎ O_{y} O_{y}, having at last one hydroxyl substituent; n is a positive integer from 1 to 11; y is zero or a positive integer less than one equal to n; each x is independently selected from the group consisting of 2, 3, 4, 5, and 6; wherein at least about 98.4% of compounds of Formula (I) in the composition have the same structure.

In an embodiment, M is Gd⁺³. In an embodiment, R₄ and R₇ are C₃H₆OH; R₅ and R are C₂H₅; R₃ and R₈ are CH₃; and RI and R₂ are H. In an embodiment, each x is 3. In an embodiment, each X is AcO⁻. In an embodiment, M is Lu⁺³. In an embodiment, R₄ and R₇ are C₃H₆OH; R₅ and R₆ are C₂H₅; R₃ and R₈ are CH₃; R₁ and R₂ are H. In an embodiment, each x is 3. In an embodiment, each X is AcO⁻. In a further or alternate embodiment, each X is selected from the group consisting of sugar derivatives, cholesterol derivatives, PEG acids, organic acids, organosulfates, organophosphates, phosphates or inorganic ligands. In a further or alternate embodiment, X is derived from an acid selected from the group consisting of gluconic acid, glucoronic acid, cholic acid, deoxycholic acid, methylphosphonic acid, phenylphosphonic acid, phosphoric acid, formic acid, propionic acid, butyric acid, pentanoic acid, 3,6,9-trioxodecanoic acid, 3,6-dioxoheptanoic acid, 2,5-dioxoheptanoic acid, methylvaleric acid, glycolic acid, pyruvic acid, oxalic acid, malic acid, malonic acid, succinic acid, maleic acid, fumaric acid, tartaric acid, citric acid, methanesulfonic acid, ethanesulfonic acid, benzoic acid, salicylic acid, 3-fluorobenzoic acid, 4-aminobenzoic acid, cinnamic acid, mandelic acid, and p-toluene-sulfonic acid. In an embodiment, the texaphyrin metal complex is motexafin gadolinium (MGd) or Xcytrin®.

Texaphyrin metal complexes are strongly absorbed in tissue, in the transparent 730-770 nm range. These complexes are capable of existing in both its free-base form and of supporting the formation of complexes with a variety of metal cations, such as Cd²⁺, Hg²⁺, In³⁺, Y³⁺, Nd³⁺, E Sm³⁺, La³⁺, Lu³⁺, Gd³⁺, and other cations of the lanthanide series.

Because of their paramagnetic property, texaphyrin-metal complexes have been used to detect tumor localization with magnetic resonance imaging (MRI). For example, Rosenthal et Clinical Cancer Research 5: 739-45 (1999) shows clinical results using Gadolinium Texaphyrin (Gd-Tex) as a tumor selective radiation sensitizer detectable by magnetic resonance imaging, and Gd-Tex shows selective biolocolization in tumors.

In addition, Viala et al, Radiology 212: 755-59 (1999), shows clinical results that Tex is tumor selective and brain metastases can be depicted at MRI long after the administration Gd-Tex. Also Carde et al, Journal of Clinical Oncology 19(7): 2074-83 (2001), shows clinical results that MGd is well tolerated at doses up to 6.3 mg/kg, is selectively accumulated in tumors, and, when combined with WBRT of 30 Gy in 10 fractions, is associated with a high radiologic response rate.

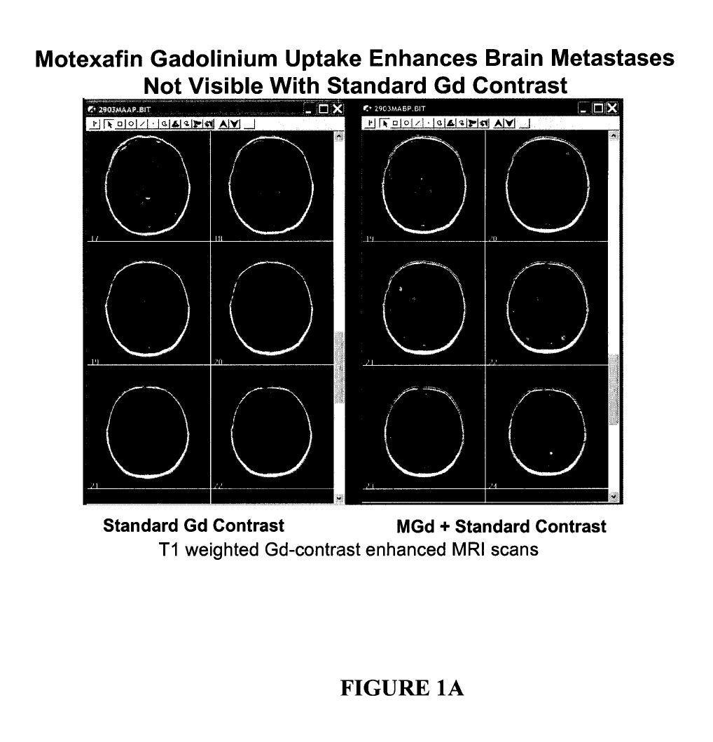

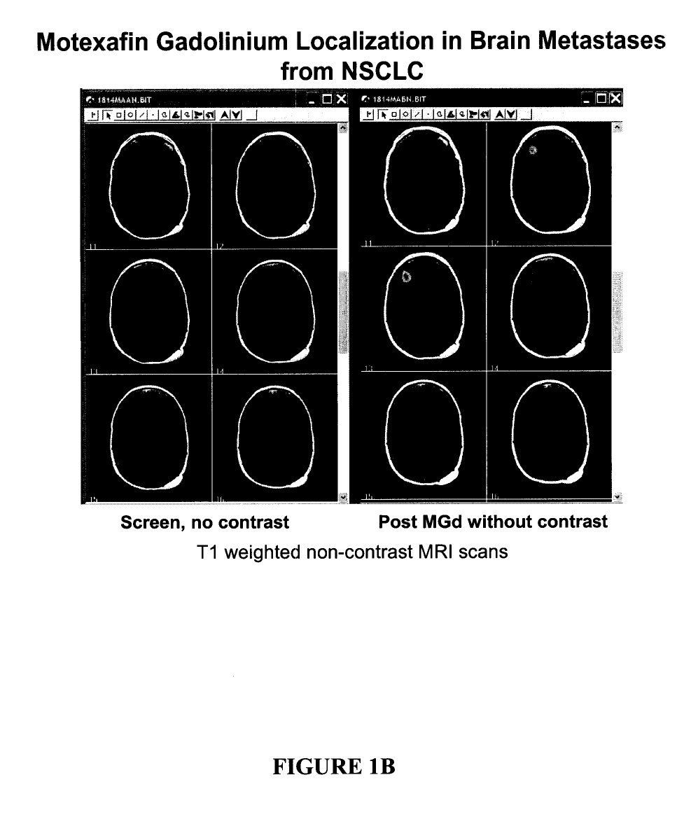

Furthermore, Mehta et al., Journal of Clinical Oncology 21(13): 2529-36 (2003), shows clinical results that MGd treatment provides benefit to patient's neurological progression including neurologic and neurocognitive function assessment in patients with brain metastases from slid tumors receiving WBRT. FIG. IA shows that MGd uptake enhances brain metastases not visible with standard Gd contrast. In particular, sections 20, 21, and 22 on the right panel (MGd+standard contrast) show brain metastases not visible in sections 20, 21, and 22 on the left panel (standard Gd contrast). FIG. 1B shows that MAGd localization in brain metastases from NSCLC. particular, sections 12, 13, and 14 on the right panel (post MGd without contrast) show brain metastases not visible in sections 12, 13, and 14 on the left panel (screen, no contrast).

Exemplary methods for synthesizing high purity texaphyrins (i.e., having less than about 1.6% polydispersity at the polyethylene glycol chain) is described, for example, in U.S. patent application Ser. No. 11/235,475. These methods provide high-purity compositions that exhibit desirable solubility, dose optimization, and drug stabilization, wherein at least about 98.4% of the compounds in the high purity compositions/samples have the same structure (and the same molecular weight, excluding isotopic variation), i.e., both polyethylene glycol chain lengths on the aromatic moiety have the same chain length.

Formulations, Routes ofAdministration, and Effective Doses

The phrase “pharmaceutically acceptable derivatives” of a compound include salts, esters, enol ethers, enol esters, acetals, ketals., orthoesters, hemiacetals, hemiketals, acids, bases, solvates, hydrates or prodrugs there of. Such derivatives may be readily prepared by those of skill this art using known methods for such derivatization. The compounds produced may be administered to animals or humans without substantial toxic effects and either can be pharmaceutically active or are prodrugs.

A “prodrug” refers to a drug or compound in which the pharmacological action results from conversion by metabolic processes within the body. Prodrugs are generally drug precursors that, following administration to a subject and subsequent absorption, are converted to an active, or a more active species via some process, such as conversion by a metabolic pathway. Some prodrugs have a chemical group present on the prodrug that renders it less active and/or confers solubility or some other property to the drug. Once the chemical group has been cleaved and/or modified from the prodrug the active drug is generated. Prodrugs may be designed as reversible drug derivatives, for use as modifiers to enhance drug transport to site-specific tissues. The design of prodrugs to date has been to increase the effective water solubility of the therapeutic compound for targeting to regions where water is the principal solvent. Prodrug forms of compounds described herein, wherein the prodrug is metabolized in vivo to produce a derivative as set forth above are included within the scope of the claims. Indeed, some of the above-described derivatives may be prodrug for another derivative or active compound.

For a more detailed discussion of exemplary prodrugs embraced within the scope of the methods described herein, see, e.g., Fedorak et al., Am. J. Physiol. 269:G210-218 (1995); McLoed et al., Gastroenterol. 106:405-413 (1994); Hochhaus et al., Biomed. Chrom. 6:283-286 (1992); Larsen et al., Int. J. Pharmaceutics 37:87 (1987); Larsen et al., Int. J. Pharmaceutics 47:103 (1988); Sinkula et al., J. Pharm. Sci. 64:181-210 (1975); Higuchi et al., Pro-drugs as Novel Delivery Systems, Vol. 14 of the A. C. S. Symposium Series, American Pharmaceutical Association and Pergamon Press (1987).

The compounds of Formula (I) may be provided as a prodrug to facilitate in vivo conversion of the prodrug (e.g., via hydrolysis, nucleophilic substitution, and the like) to a compound having desired biological activity at some time after administration.

Compositions employed according to methods presented herein comprise at least one compound of Formula (I) and, optionally, a pharmaceutically acceptable salt.

The term “pharmaceutically acceptable salt” means those salts which retain the biological effectiveness and properties of the compounds disclosed herein, and which are not biologically or otherwise undesirable. For example, a pharmaceutically acceptable salt does not interfere with the beneficial effect of the compound disclosed herein in treating a cancer.

Typical salts include by way of example only, salts formed by mixing a compound of Formula (I) in an appropriate buffer, such as phosphate buffer, or by passing a compound of Formula (I) through an appropriate ion-exchange column. In addition, if the compounds disclosed herein contain a carboxy group or other acidic group, it may be converted into a pharmaceutically acceptable addition salt with inorganic or organic bases.

Examples of pharmaceutically acceptable salts include acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid. mandelic acid, methanesulfonic acid, ethanesulfonic acid, 1,2-ethanedisulfonic acid, 2-hydroxyethbanesulfonic acid, benzenesulfonic acid, 2-naphthalenesulfonic acid, 4-methylbicyclo-[2.2.2]oct-2-ene-1-carboxylic acid, glucoheptonic acid 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

Examples of pharmaceutically acceptable salts also include salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine, tromethamine, N-methylglucamine, and the like. Acceptable inorganic bases include aluminum hydroxide, calcium hydroxide, potassium hydroxide, sodium carbonate, sodium hydroxide, and the like. It should be understood that a reference to a pharmaceutically acceptable salt includes the solvent addition forms or crystal forms there of, particularly solvates or polymorphs. Solvates contain either stoichiometi or non-stoichiometric amounts of a solvent, and may be formed during the process of crystallization. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

Pharmaceutically acceptable esters include, but are not limited to, alkyl, alkenyl, alkynyl, aryl, heteroaryl, aralkyl, heteroaralkyl, cycloalkyl and heterocyclyl esters of acidic groups including, but not limited to, carboxylic acids, phosphoric acids, phosphinic acids, sulfonic acids, sulfinic acids and boronic acids. Pharmaceutically acceptable enol ethers include, but are not limited to, derivatives of formula C=C(OR) where R is hydrogen, alkyl, alkenyl, alkynyl, aryl, heteroaryl, aralkyl, heteroaralkyl, cycloalkyl or heterocyclyl. Pharmaceutically acceptable enol esters include but are not limited to, derivatives of formula C=C(OC(O)R) where R is hydrogen, alkyl, alkenyl, alkynyl, aryl, heteroaryl, aralkyl, heteroaralkyl, cycloalkyl or heterocyclyl. Pharmaceutically acceptable solvates and hydrates are complexes of a compound with one or more solvent or water molecules, or 1 to about 100, or 1 to about 10, or one to about 2, 3 or 4, solvent or water molecules.

Compositions employed according to methods presented herein comprise at least one compound of Formula (I) and, optionally, at least one pharmaceutically acceptable excipient, carris adjuvant, and the like.

Selection of a suitable excipient, carrier, and/or adjuvant will depend on the route administration contemplated. For example, the excipient can be a liquid suited for administration injection, including intravenous, intramuscular, or subcutaneous administration. Alternatively, the excipient can be suited to topical, transdermal, or buccal administration, or as a suppository.

Suitable excipients or carriers are, for example, water, saline, dextrose, glycerol, mannitol, acids (e.g., acetic acid), buffers, alcohols, aloe vera gel, allantoin, glycerin, vitamin A and E oils, mineral oil, propylene glycol, PPG-2 myristyl propionate, and the like. These compositions may also contain minor amounts of nontoxic, auxiliary substances, such as wetting or emulsifying. agents, pH buffering agents, and so forth.

Suitable storage-stabilized formulations of texaphyrin metal complexes include water and acetic acid. In one embodiment, the storage-stabilized formulation should have a pH of 5.4. In other embodiments, the storage-stabilized formulation should have a pH between about 4.5-5.5, about 5.0-5.9 or about 4.9-5.9. In another embodiment the concentration of the texaphyrin metal complex in the storage-stabilized formulation is between 2.2 mg/mL and about 3.0 mg/mL; in a further embodiment the concentration of the texaphyrin metal complex is between about 2.5 mg/mL and about 3.0 mg/mL; in a further embodiment the concentration of the texaphyrin metal complex is about 2.5 mg/mL.

In further or alternative embodiments, storage-stabilized formulation contains an isotonic agent, which can include electrolytes and/or non-electrolytes. Non-limiting examples of electrolytes includes sodium chloride, potassium chloride, dibasic sodium phosphate, sodium gluconate and combinations there of. Non-limiting examples of non-electrolytes includes saccharides and polyhydric alcohols; further examples include mannitol, sorbitol, glucose, dextrose, glycerol, xylitol, fructose, maltose, mannose, glycerin, propylene glycol, and combinations there of. In still further embodiments, the storage-stabilized formulation comprises a buffer, an anti-crystallizing agent, and/or a preservative. Buffering agents aid in stabilizing pH. Anti-crystallizing agents aid in stabilizing the concentration of the solution. Preservatives aid in preventing the growth of micro-organisms, and include by way of example only, methyl paraben, propyl paraben, benzyl alcohol, sodium hypochlorite, phenoxy ethanol and/or propylene glycol. In one, the storage-stabilized formulation does not contain an oxidizing agent other than the texaphyrin metal complex and oxygen. Oxidizing agents promote degradation of the texaphyrin metal complex.

A carrier can be one or more substances which also serve to act as a diluent, flavoring agent, solubilizer, lubricant, suspending agent, binder, or tablet disintegrating agent. A carrier can also be an encapsulating material.

In powder forms, the carrier is preferably a finely divided solid in powder form that interdispersed as a mixture with a finely divided powder from of one or more compound. In table forms of the compositions, one or more compounds is intermixed with a carrier with appropriate binding properties in suitable proportions followed by compaction into the shape and size desired. Powder and tablet form compositions preferably contain between about 5 to about 70% by weight one or more compound. Carriers that may be used in the practice include, but are not limited to, magnesium carbonate, magnesium stearate, talc, lactose, sugar, pectin, dextrin, starch, tragacanth, methyl cellulose, sodium carboxymethyl cellulose, a low-melting wax, cocoa butter, and the like.

Carriers also include any commonly used excipients in pharmaceutics and should be selected on the basis of compatibility with the compounds disclosed herein and the release pr ofile properties of the desired dosage form. Exemplary carriers include, e.g., binders, suspending agents disintegration agents, filling agents, surfactanits, solubilizers, stabilizers, lubricants, wetting agents diluents, and the like. Pharmaceutically acceptable carriers may comprise, e.g., acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, and the like.

The compounds described herein may also be encapsulated or microencapsulated by an encapsulating material, which may thus serve as a carrier, to provide a capsule in which the derivatives, with or without other carriers, is surrounded by the encapsulating material. In an analogous manner, cachets comprising one or more compounds are also provided. Tablet, powder capsule, and cachet forms of the may be formulated as single or unit dosage forms suitable for administration, optionally conducted orally. For intravenous injections, the compounds described herein may be formulated in aqueous solutions, preferably in physiologically compatible buffers such as Hank's solution, Ringer's solution, or physiological saline buffer.

In suppository forms of the compositions, a low-melting wax such as, but not limited to, a mixture of fatty acid glycerides, optionally in combination with cocoa butter is first melted. One or more compounds are then dispersed into the melted material by, as a non-limiting example stirring. The non-solid mixture is then placed into molds as desired and allowed to cool and solid

Non-limiting compositions in liquid form include solutions suitable for oral, injection or parenteral administration, as well as suspensions and emulsions suitable for oral administration Sterile aqueous based solutions of one or more compounds, optionally in the presence of an agent increase solubility of the derivative(s), are also provided. Non-limiting examples of sterile solutions include those comprising water, ethanol, and/or propylene glycol in forms suitable for parenteral administration. A sterile solution comprising a compound described herein may be prepared by dissolving one or more compounds in a desired solvent followed by sterilization, such as by filtration through a sterilizing membrane filter as a non-limiting example. In another embodiment, one or more compounds are dissolved into a previously sterilized solvent under sterile conditions

A water based solution suitable for oral administration can be prepared by dissolveing one or more compounds in water and adding suitable flavoring agents, coloring agents, stabilizers and thickening agents as desired. Water based suspensions for oral use can be made by dispersing one or more compounds in water together with a viscous material such as, but not limited to, natural or synthetic gums, resins, methyl cellulose, sodium carboxymethyl cellulose, and other suspending agents known to the pharmaceutical field.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methylcellulose. The formulations can additionally include: lubricating agents such as talc, magnesium stearate, and mineral oil; wetting agents; emulsifying and suspending agents; preserving agents such as methyl-and propylhydroxy-benzoates; sweetening agents; and flavoring agents.

At high concentrations, texaphyrin metal complexes have a tendency to aggregate in aqueous solution, which potentially decreases their solubility. Aggregation may significantly alter the photochemical characteristics of the macrocycles in solution, which is shown by large spectral changes, decrease in extinction coefficient, etc. Addition of a carbohydrate, saccharide, polysaccharide, or polyuronide to the formulation decreases the tendency of the texaphyrin to aggregate, thus increasing the solubility of the texaphyrin in aqueous media. Examples of such agents are sugars, including mannitol, dextrose or glucose. In one embodiment, mannitol is used concentrations of about 2-8% concentration, including about 5% concentration. These aqueous solutions are suitable for intravenous, intramuscular, subcutaneous and intraperitoneal administration.

Compositions according to methods presented herein can be administered to a patient in any pharmaceutically acceptable route or manner.

One mode for administration is parenteral, including, by way of example, by injection. The forms in which the high-purity compositions comprising compounds of Formula (I) may be incorporated for administration by inj ection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection. Ethanol, glycerol, propylene glycol, liquid polyethylene glycol, and the like (and suitable mixtures there of), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol phenol, sorbic acid, thimerosal, and the like.

Sterile injectable solutions are prepared by incorporating the high-purity compositions of Formula (I) in the required amount in the appropriate solvent with various other ingredients as enumerated above, as required, followed by sterile filtration. Generally, dispersions are prepared by incorporating the various sterilized high-purity compositions comprising compounds of Formula (I) into a sterile vehicle which contains the basic dispersion medium and the required other ingredients from those enumerated above. In the case of sterile powders for the preparation of sterile injectable solutions, the methods of preparation are vacuum-drying and freeze-drying techniques, which yield a powder of the high-purity compositions comprising compounds of Formula (I) plus any additional desired ingredient from a previously sterile-filtered solution there of

The high-purity compositions comprising compounds of Formula (I) may be impregnated into a stent by diffusion, for example, or coated onto the stent such as in a gel form, for example, using procedures known to one of skill in the art in light of the present disclosure.

Oral administration is another route for administration of the high-purity compositions comprising compounds of Fornula (I). Embodiments include oral administration capsule or enteric-coated tablets, or the like, which prevent degradation in the stomach. Compositions can be in the form of tablets, pills, powders, lozenges, sachets, cachets, elixirs, suspensions, emulsions, solutions, syrups, aerosols (as a solid or in a liquid medium), ointments, sol and hard gelatin capsules, sterile injectable solutions, and sterile packaged powders.

The high-purity compositions comprising compounds of Formula (I) can be fonmulated so as to provide quick, sustained or delayed release after administration to the patient by employing well known procedures known in the art. Controlled release drug delivery systems for oral administration include osmotic pump systems and dissolutional systems containing polymer-coated reservoirs or drug-polymer matrix fonnulations. Examples of controlled release systems and given in U.S. Pat. Nos. 3,845,770; 4,326,525; 4,902,514; and 5,616,345.

The tablets or pills described herein may be coated or otherwise compounded to provide a dosage form affording the advantage of prolonged action, or to protect from the acid conditions of the stomach. For example, the tablet or pill can comprise an inner dosage and an ou dosage component, the latter being in the fonn of an envelope over the fonmer. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate

Compositions comprising compounds of Fonmula (I) may be administered using transdermal delivery devices (“patches”). Such transdermal patches may be used to provide continuous or discontinuous infusion in controlled amounts. See, e.g., U.S. Pat. Nos. 5,023,252; 4,992,445; and 5,001,139. Such patches may be constructed for continuous, pulsatile, or on demand delivery of pharmaceutical agents.

Compositions for inhalation or insufflation include solutions and suspensions in pharmaceutically acceptable, aqueous or organic solvents, or mixtures there of, and powders. The liquid or solid compositions may contain suitable pharmaceutically acceptable excipients as described herein. Such compositions are administered by the oral or nasal respiratory route for local or systemic effect. Compositions in pharmaceutically acceptable solvents may be nebulized by use of inert gases. Nebulized solutions may be inhaled directly from the nebulizing device or the nebulizing device may be attached to a facemask tent, or intermittent positive pressure-breathing machine. Solution, suspension, or powder compositions may be administered, orally or nasally, from devices that deliver the formulation in an appropriate manner.

Compositions comprising compounds of Formula (I) are optionally formulated in a unit dosage form. The term “unit dosage forrn(s)” refers to physically discrete units suitable as unitary dosages (e.g., a tablet, capsule, ampule) for human subjects and other mammals, each unit containing a predetermined quantity of active material calculated to produce the desired therapeut effect, in association with a suitable pharmaceutical excipient.

The high-purity compositions comprising compounds of Formula (I) are effective over a wide dosage range and are generally administered in an amount effective to achieve a desired biological effect. Appropriate guidelines for (lose amounts are provided herein. Exact amounts of the compound administered will be determined by a physician, in the light of the relevant circumstances, including the condition to be treated, the chosen route of administration, the actual compound administered and its relative activity, the age, weight, and response of the individual patient, the severity of the patient's symptoms, and the like. Specific doses will vary depending on the compound chosen, the purity of the composition, the dosing regimen to be followed, and the particular therapeutic energy or agent with which the compound is administered. There are special differences in the most effective dosimetry depending on the apical ligands chosen, because of the wide range of properties available, such as solubilities, lipophilicity properties, lower toxicity, and improved stability.

Treating Cancers

Without limiting the scope of the compositions and the methods disclosed herein, the methods are used to treat various cancers or tumors. Cancer types include (some of which may overlap in scope), by way of example only, adrenal cortical cancer, anal cancer, aplastic anemia, duct cancer, bladder cancer, bone cancer, borne metastasis, adult CNS brain tumors, pediatric CNS brain metastases, brain metastases, breast cancer, Castleman Disease, cervical cancer, childhood Non-Hodgkin's lymphoma, colon and rectum. cancer, endometrial cancer, esophagus cancer, Ewing's family of tumors, eye cancer, gallbladder cancer, gastrointestinal carcinoid tumors, gastrointestinal stromal tumors, gestational triophoblastic disease, hematological malignancies, Hodgkin's disease, Kaposi'sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, nonmelanoma skin cancer, stomach cancer, testicular cancer, thymus cancer, thyroid cancer, uterine sacrcoma, vaginal cancer, vulvar cancer, and Waldenstrom's macroglobulinemia. In one embodiment, the cancers are selected from the group consisting of metastatic brain cancer, lung cancer, glioblastoma, lymphomas, leukemia, renal cell cancer (kidney cancer), head and neck cancer, breast cancer, prostrate cancer, and ovarian cancer.

Disclosed herein are methods of treating lung cancer by administering a therapeutically effective amount of at least one texaphyrin metal complex or a pharmaceutically acceptable derivative, and performing stereotactic radiosurgery to the patient. Treatment options lung cancer include (which can be provided to a patient in conjunction with the methods disclosed herein), by way of example only, surgery, immunotherapy, radiation therapy, chemotherapy, photodynamic therapy, or a combination there of. Some possible surgical options for treatment of lung cancer are a segmental or wedge resection, a lobectomy, or a pneumonectomy. Radiation therapy may be external beam radiation therapy or brachytherapy.

Disclosed herein are methods of treating CNS neoplasms by administering a therapeutically effective amount of at least one texaphyrin metal complex or a pharmaceutically acceptable derivative, and performing stereotactic radiosurgery to the patient. Treatment options CNS neoplasms include (which can be provided to a patient in conjunction the methods disclosed herein), by way of example only, surgery, radiation therapy, immunotherapy, hyperthermia, gene therapy, chemotherapy, and combination of radiation and chemotherapy. Doctors also may prescribe steroids to reduce the swelling inside the CNS.

Disclosed herein are methods of treating kidney cancer by administering a therapeutically effective amount of at least one texaphyrin metal complex or a pharmaceutically acceptable derivative, and performing stereotactic radiosurgery to the patient. Kidney cancer (also called renal cell cancer or renal adenocarcinoma) is a disease in which malignant cells are found in the lining of tubules in the kidney. Treatment options for kidney cancer include (which can be provided to a patient in conjunction with the methods disclosed herein), by way of example only, surgery, radiation therapy, chemotherapy and immunotherapy. Some possible surgical options to treat kidney cancer include, by way of example only, partial nephrectomy, simple nephrectomy and radical nephrectomy. Radiation therapy may be external beam radiation therapy or brachytherapy. Stem cell transplant may be used to treat kidney cancer.

Disclosed herein are methods of treating lymphoma by administering a therapeutically effective amount of at least one texaphyrin metal complex or a pharmaceutically acceptable derivative, and performing stereotactic radiosurgery to the patient. Treatment options lymphoma include (which can be provided to a patient in conjunction with the methods disclosed herein), by way of example only, chemotherapy, immunotherapy, radiation therapy and high-dose chemotherapy with stem cell transplant. Radiation therapy may be external beam radiation therap or brachytherapy.

Disclosed herein are methods of treating breast cancer by administering a therapeutically effective amount of at least one texaphyrin metal complex or a pharmaceutically acceptable derivative, and performing stereotactic radiosurgery to the patient. Treatment options breast cancer include (which can be provided to a patient in conjunction with the methods disclosed herein), by way of example only, surgery, immunotherapy, radiation therapy, chemotherapy, endocrine therapy, or a combination there of. A lumpectomy and a mastectomy are two possible surgical procedures available for breast cancer patients.

Disclosed herein are methods of treating ovarian cancer by administering a therapeutically effective amount of at least one texaphyrin metal complex or a pharmaceutically acceptable derivative, and performing stereotactic radiosurgery to the patient. Treatment options to ovarian cancer include (which can be provided to a patient in conjunction with the methods disclosed herein), by way of example only, surgery, immunotherapy, chemotherapy, hormone therapy, radiation therapy, or combinations there of. Some possible surgical procedures include debulking, and a unilateral or bilateral oophorectomy and/or a unilateral or bilateral salpigectomy.

Disclosed herein are methods of treating cervical cancer by administering a therapeutically effective amount of at least one texaphyrin metal complex or a pharmaceutically acceptable derivative, and performing stereotactic radiosurgery to the patient. Treatment options for cervical cancer include (which can be provided to a patient in conjunction with the methods disclosed herein), by way of example only, surgery, immunotherapy, radiation therapy and chemotherapy. Some possible surgical options are cryosurgery, a hysterectomy, and a radical hysterectomy. Radiation therapy for cervical cancer patients includes external beam radiation therapy or brachytherapy.

Disclosed herein are methods of treating prostate cancer by administering a therapeutically effective amount of at least one texaphyrin metal complex or a pharmaceutically acceptable derivative, and performing stereotactic radiosurgery to the patient. Treatment options for prostate cancer include (which can be provided to a patient in conjunction with the methods disclosed herein), by way of example only, surgery, immunotherapy, radiation therapy, cryosurgery, hormone therapy, and chemotherapy. Possible surgical procedures to treat prostate cancer included by way of example only, radical retropubic prostatectomy, a radical perineal prostatectomy, and a laparoscopic radical prostatectomy. Some radiation therapy options are external beam radiation, including three dimensional conformal radiation therapy, intensity modulated radiation therapy, and conformal proton beam radiation therapy. Brachytherapy (seed implantation or interstitial radiation therapy) is also an available method of treatment for prostate cancer. Cryosurgery is another possible method used to treat localized prostate cancer cells. Hormone therapy, also called androl deprivation therapy or androgen suppression therapy, may be used to treat prostate cancer. Several methods of this therapy are available including an orchiectomy in which the testicles, where 90% androgens are produced, are removed. Another method is the administration of luteinizing hormone releasing hormone (LHRH) analogs to lower androgen levels. The LHRH analogs available includs leuprolide, nafarelin, goserelin, triptorelin, and histrelin. An LHRH antagonist may also be administered, such as abarelix. Treatment with an antiandrogen agent, which blocks androgen activity in the body, is another available therapy. Such agents include flutamide, bicalutamide, and nilutamide. This therapy is typically combined with LHRH analog administration or an orchiectomy, which is termed a combined androgen blockade (CAB). Chemotherapy may be appropriate where a prostate tumor has spread outside the prostate gland and hormone treatment is not effective. Anti-cancer drugs may be administered to slow the growth of prostate cancer, reduce symptoms and improve the quality of life.

Disclosed herein are methods of treating leukemia by administering a therapeutically effective amount of at least one texaphyrin metal complex or a pharmaceutically acceptable derivative, and performing stereotactic radioesurgery to the patient. Treatment options for leukemia include (which can be provided to a patient in conjunction with the methods disclosed herein), by way of example only, immunotherapy, radiation therapy, chemotherapy, bone marrow or periphera blood stem cell transplantation, or a combination there of. Radiation therapy includes external bean radiation and may have side effects. Anti-cancer drugs may be used in chemotherapy to treat leukemia. Monoclonal antibody therapy may be used to treat AML patients. Small molecules or radioactive chemicals may be attached to these antibodies before administration to a patient in order to provide a means of killing leukemia cells in the body. The monoclonal antibody, gemtuzumab ozogamicin, which binds CD33 on AML cells, may be used to treat AML patients unable to tolerate prior chemotherapy regimens. Bone marrow or peripheral blood stem cell transplantation may be used to treat AML patients. Some possible transplantation procedures are an allogenic or an autologous transplant.

Disclosed herein are methods of treating head and neck cancer by administering a therapeutically effective amount of at least one texaphyrin metal complex or a pharmaceutically acceptable derivative, and performing stereotactic radiosurgery to the patient. Treatment options head and neck cancer include (which can be provided to a patient in conjunction with the methods disclosed herein), by way of example only, surgery, radiation, chemotherapy, combined modality therapy, monoclonal antibody treatment, irnriunotherapy, gene therapy, either alone or in combination there of.

The methods as described herein, may be combined with administered before, at the same time as, or after administration of one or more chemotherapeutic drugs. At least one texaphyrin-metal complex or a pharmaceutically acceptable derivative may be administered concurrently with, or from about 1 minute to about 12 hours following administration of a chemotherapeutic drug, preferably from about 5 minutes to about 5 hours, more preferably about 4 to 5 hours. The dosing protocol may be repeated, from one to three times, for example.

A “chemotherapeutic agent” may be, but is not limited to, one of the following: an alkylating agent such as a nitrogen mustard, an ethyleneimine or a methylmelamine, an alkyl sulfonate, a nitrosourea, or a triazene; an anti-metabolite such as a folic acid analog, a pyrimidine analog, or a purine analog; a natural product such as a vinca alkaloid, an epipodophyllotoxin, an antibiotic, an enzyme, taxane, or a biological response modifier; miscellaneous agents such as a platinum coordination complex, an anthracenedione, an anthracycline, a substituted urea, a methy hydrazine derivative, or an adrenocortical suppressant; or a hormone or an antagonist such as an adrenocorticosteroid, a progestin, an estrogen, an antiestrogen, an androgen, an antiandrogen, or a gonadotropin-releasing hormone analog. Chemotherapeutic agents are used in the treatment of cancer and other neoplastic tissue. Preferably, the chemotherapeutic agent is a nitrogen mustard, an epipodophyllotoxin, an antibiotic, or a platinum coordination complex. A more preferred chemotherapeutic agent is bleomycin, doxorubicin, taxol, taxotere, etoposide, 4-OH cyclophosphamide, cisplatin, or platinum coordination complexes analogous to cisplatin. A presently preferred chemotherapeutic agent is doxorubicin, taxol, taxotere, cisplatin, or Pt complexes analogous to cisplatin. Various chemotherapeutic agents, their target diseases, and treatment protocols are presented in, for example, Goodman and Gilman 's The Pharmacological Basis of Therapeutics, Ninth Ed., Pergamon Press, Inc., 1990; and Remington: The Science and Practice of Pharmacy, Mack Publishing Co., Easton, Pennsylvania., 1995; both of which are incorporated by reference herein.

At least one texaphyrin-metal complex or a pharmaceutically acceptable derivative may be administered before, at the same time, or after administration of one or more chemotherapeutic drugs. At least one texaphyrin-metal complex or a pharmaceutically acceptable derivative may be administered concurrently with, or from about one minute to about 12 hours following, administration of a chemotherapeutic drug, preferably from about 5 min to about 5 hr, more preferably about 4 to 5 hr. The dosing protocol may be repeated, from one to three times, for example. Administration may be intra-arterial injection, intravenous, intraperitoneal, intramuscular subcutaneous, oral, topical, or via a device such as a stent, for example, with parenteral and intra-arterial administration being preferred, and intra-arterial being more preferred.

In further applications, the pharmaceutical composition comprising texaphyrin metal complexes as described herein, where M is Gd⁺³, may be administered with NAD(P)H, ascorbate and other reducing agents under approximate physiological conditions, leading to reactive oxygen species generation. Depletion of these reducing agents will inhibit biochemical pathways that in vivo utilize reducing agents to effect repair of the damage inflicted by reactive oxygen species. S a method may be used to treat cancer and cardiovascular diseases. See U.S. Pat. No. 6,825,186 which is incorporated by reference in its entirety.

An embodiment presented herein is directed to methods of detecting, quantifying, identifying, modulating, and/or treating metastases comprising administering an effective amount at least one high-purity texaphyrin metal complex, such as motexafin gadolinium (MGd), to a patient, and performing stereotactic radiosurgery on a patient's brain. Methods herein can be usesd to treat patients exhibiting metastases from a solid tumor, such as, but not limited to, lung cancer cel metastasized in the brain. Methods herein can be used to treat patients exhibiting metastases from non-solid tumors, such as in hematological cancers.

In an embodiment, methods herein can be used to treat patients exhibit 1 to 4 metastatic sites. For example, methods herein can be used to treat patients exhibiting 1 to 4 brain metastases from a solid tumor. In an embodiment, methods herein are employed to reduce the size of metastases in patients by at least about 25%/o, such as about 50%, about 60%, about 75%, about 85% and about 95%.

One aspect disclosed herein are methods of modulating brain metastases comprising administering an effective amount of at least one high-purity texaphyrin metal complex, such as motexafin gadolinium (MGd), to a patient, and performing stereotactic radiosurgery on a patient's brain. In an embodiment, the high-purity texaphyrin metal complex is administered before, during, or after stereotactic radiosurgery is performed. In another embodiment, methods herein further comprise performing whole brain radiation therapy before, during, or after administration of the high-purity texaphyrin metal complex. Methods herein can be used to treat patients exhibiting 1 to 4 brain metastases from a solid tumor. In an embodiment, methods herein are employed to reduce the size of brain metastases in patients by at least about 25%, such as about 50%, about 60%, about 75%, about 85% and about 95%. In another embodiment, methods herein are employed to reduce neurological progression in patients by at least about 25%, such as about 50%, about 60%, about 75%, about 85% and about 95%. In yet another embodiment, methods herein are employed to reduce radiological progression in patients by at least 25%, such as about 50%, about 60%, about 75%, about 85% and about 95%.

Another aspect disclosed herein are improved methods for modulating brain metastases in a patient undergoing whole brain radiation therapy prior to receiving stereotactic radiosurgery, wherein the improvement is administering an effective amount of at least one high-purity texaphyrin metal complex, such as MGd, to the patient. Improved methods herein can be used to treat patients exhibiting 1 to 4 brain metastases from a solid tumor. In an embodiment, improved methods herein are employed to reduce the size of brain metastases in patients by at lea about 25%, such as about 50%, about 60%, about 75%, about 85% and about 95%. In another embodiment, improved methods herein are employed to reduce neurological progression in patients by at least about 25%, such as about 50%, about 60%, about 75%, about 85% and about 95%. In another embodiment, improved methods herein are employed to reduce radiological progression in patients by at least about 25%, such as about 50%, about 60%, about 75%, about 85% and about 95%.

Another aspect disclosed herein are methods of modulating metastases comprising administering an effective amount of at least one high-purity texaphyrin metal complex, such as MGd, to the patient, and performing stereotactic radiosurgery on the area of a patient's body comprising the metastases. In an embodiment, the high-purity texaphyrin metal complex is administered before, during, or after stereotactic radiosurgery is performed. In another embodiment methods herein further comprise performing whole body radiation therapy before, during, or after administration of the high-purity texaphyrin metal complex. Methods herein can be used to treat patients exhibiting I to 4 metastases from a solid tumor. In an embodiment, methods herein are employed to reduce the size of the metastases in patients by at least about 25%, such as about 50%, about 60%, about 75%, about 85% and about 95%. In another embodiment, methods herein are employed to reduce neurological progression in patients by at least about 25%, such as about 50% about 60%, about 75%, about 85% and about 95%. In yet another embodiment, methods herein are employed to reduce radiological progression in patients by at least about 25%, such as about 50%. about 60%, about 75%, about 85% and about 95%.

Another aspect disclosed herein are methods for modulating various cancers comprising administering an effective amount of at least one high-purity texaphyrin metal complex such as MGd, to the patient, and performing stereotactic radiosurgery on the area of a patient's body comprising the cancer. In an embodiment, the high-purity texaphyrin metal complex is administered before, during, or after stereotactic radiosurgery is performed. In another embodiment, methods herein further comprise performing at least one alternative radiation therapy procedure, which is the same or different from stereotactic radiosurgery, before, during, or after administration of the high purity texaphyrin metal complex. Methods herein can be employed to reduce the size of the cancer in patients, such as by at least about 25%, such as about 50%, about 60%, about 75%, about 85% and about 95%. In another embodiment, methods herein are employed to reduce neurological progression in patients, such as by at least about 25%, such as about 50%, about 60%, about 75%, about 85% and about 95%. In yet another embodiment, methods herein are employed to reduce radiological progression in patients, such as by at least about 25%, such as about 50%, about 60% about 75%, about 85% and about 95%.

Yet another aspect disclosed herein are methods for detecting at least one cancerous tumor, lesion, or metastases in a patient's brain by administering an effective amount of at least one high-purity texaphyrin metal complex, such as MGd, before the patient undergoes stereotactic radiosurgery; screening the patient's brain; and detecting the presence of the texaphyrin metal complex, thereby detecting the presence of the tumor, lesion, or metastases. In an embodiment, methods herein further comprise performing whole brain radiation therapy on the patient before, during, or after administration of the high-purity texaphyrin metal complex. Methods herein can be employed to improve detection of tumors, lesions or metastases, as compared to methods which do not involve administration of a high-purity texaphyrin metal complex, such as methods which employ standard metal contrasting agents. Detection of tumors or lesions are improved using methods presented herein by at least about 25%, such as about 50%, about 60%, about 75%, about 85% and about 95%. Methods herein may be used to determine the presence, location, at least one identifying feature (e.g., shape, density, and the like), and/or size (e.g., area, width, volume, and the like) of at least one tumor, lesion, or metastases.

Another aspect disclosed herein are improved methods for detecting brain metastases in a patient, wherein the improvement is administering an effective amount of at least one high-purity texaphyrin metal complex, such as MGd, before the patient undergoes stereotactic radiosurgery. In an embodiment, improved methods herein further comprise performing whole brain radiation therapy on the patient before, during, or after administration of the high-purity texaphyrin metal complex. Detection of tumors, lesions, or metastases are improved by at least about 25%, such as about 50%, about 60%, about 75%, about 85% and about 95%. Improved methods herein may be used to determine the presence, location, at least one identifying feature (e.g., shape, density and the like), and/or size (e.g., area, width, volume, and the like) of at least one tumor, lesion, or metastases.

Another aspect disclosed herein are methods for detecting various cancerous tumors lesions, or metastases in a patient by administering an effective amount of at least one high-purity texaphyrin metal complex, such as MGd, before the patient undergoes stereotactic radiosurgery; screening the area of the patient's body comprising the tumors, lesions, or metastases; and detecting the presence of high-purity texaphyrin metal complex, thereby detecting the presence of the tumor lesion, or metastases. In an embodiment, methods herein further comprise performing at least one alternative radiation therapy, such as whole body radiation, on the patient before, during, or after administration of the high-purity texaphyrin metal complex. Methods herein can be employed to improve detection of tumors, lesions, or metastases, as compared to methods which do not involve administration of a high-purity texaphyrin metal complex, such as methods which employ standard metal contrasting agents. Detection of tumors, lesions, or metastases are improved using methods presented herein by at least about 25%, such as about 50%, about 60%, about 75%, about 85% and about 95%. Methods herein may be used to determine the presence, location, at least one identifying feature (e.g., shape, density, and the like), and/or size (e.g., area, width, volume, and the like) of at least one tumor, lesion, or metastases.

Another aspect disclosed herein are improved methods for detecting various cancerous tumors, lesions, or metastases, wherein the improvement is administering an effective amount of least one high-purity texaphyrin metal complex, such as MGd, prior to performing stereotactic radiosurgery on the patient. In an embodiment, improved methods herein further comprise performing at least one alternative radiation therapy, such as whole body radiation, on the patient before, during, or after administration of the high-purity texaphyrin metal complex. Detection of tumors, lesions, or metastases are improved by at least about 25%, such as about 500%, about 60%, about 75%, about 85% and about 95%. Improved methods herein may be used to determine the presence, location, at least one identifying feature (e.g., shape, density, and the like), and/or size (e.g., area, width, volume, and the like) of at least one tumor, lesion, or metastases.

### EXAMPLES

The following illustrative examples are representative embodiments of the invention, and are not meant to be limiting in any way.

### Example 1

### Phase II Trial of Motexafin Gadolinium with Radiation therapy Followed by Stereotactic Radiosurgery Boost

A Phase II one-arm, open-label study to evaluate the safety and potential efficacy treatment with MGd administered concomitantly with whole brain radiation therapy (WBRT) and stereotactic radiosurgery (SRS boost) for the treatment of brain metastases from solid tumors is conducted. This clinic trial also evaluates brain lesion number and size after 11 doses of MGd compared with those visualized by the use of standard MRI contrast agents. Provided below is a brief synopsis of various aspects of the clinical study is provided in Table 1 below.

TABLE 1
Summary of Protocol for Phase II Clinical Study

| | |
|---|---|
| Indication | Brain metastases from solid tumors |
| Study Duration | Patients receive 15 doses of WBRT over 3 weeks, that is |
| | administered concurrently with MGd during the second and third |
| | week of treatment. Subsequent single treatment of MGd is given on |
| | the day of the SRS boost (to be given within 14 days of WBRT |
| | completion). Survival, neurologic progression, and radiologic |
| | progression is monitored in patients until death or until 6 months |
| | after the 45^{th} evaluable patient has enrolled. |
| Study Population | Approximately 45 patients with 1 to 4 brain metastases from solid |
| | tumors |
| Recorded | Primary: Rate of irreversible Grade 3 or any Grade 4 or 5 |
| Endpoints | neurologic radiation toxicities occurring within 3 months following |
| | SRS boost. |
| | Secondary: |
| | Change in lesion size and number between screening MRI and |
| | SRS treatment-planning MRI |
| | Time to neurologic progression or death with evidence of |
| | neurologic progression. |
| | Time to neurocognitive progression |
| | Time to all-cause mortality |
| | Exploratory: |
| | Time to radiologic progression |
| | Radiologic response rate at Month 3 |
| Investigational | WBRT: Administered 5 days/week for 15 doses during Weeks 1 |
| Drug, Dose, Route, | through 3 (37.5 Gy in 15 fractions). |
| Regimen | MGd: 10 doses of MGd 5 mg/kg infused intravenously once a day, |
| | 5 days/week beginning with the 6^{th} dose of WBRT, 2 to 5 hours |
| | before each WBRT fraction of 2.5 Gy (fractions 6 to 15). During |
| | Week 4 (preferred) or Week 5, MGd 5 mg/kg followed a minimum |
| | of 2 hours later by SRS boost. |
| | SRS boost: Once within 14 days of the last WBRT treatment, with |
| | the dose for each lesion to be determined by lesion size: 21.0 Gy |
| | (lesions ≦2.0 cm), 18.0 Gy (lesions >2.0 cm and ≦3.0 cm) or 15.0 |
| | Gy (lesions >3.0 cm and ≦4.0 cm). Up to 6 lesions may be treated |
| | to continue with the study. A SRS dose reduction of up to 25% is |
| | permitted at Investigator's discretion in patients with multiple |
| | lesions, if these lesions are believed to either be too close to each |
| | other, or to critical normal structures. If more than 6 lesions exist at |
| | the time of SRS it is recommended that SRS not be done. At the |
| | time of this treatment premedication with antiemetics is given. |
| Visit/Treatment | Treatment visits: During the first week of treatment patients |
| Schedule | receive five dose of WBRT (2.5 Gy each). During Weeks 2 and 3 |
| | patients receive 10 doses of WBRT and 10 doses of MGd. |
| | Within 14 days following the last WBRT dose patients are treated |
| | with MGd and SRS. If the treatment-planning MRI takes place prior |
| | to the SRS day and more than 4 days following the last dose of |
| | MGd, then an additional dose of MGd is administered prior to the |
| | MRI. |
| | Follow-up: Monthly through Month 9 (from start of WBRT) and in |
| | 3-month intervals thereafter. Confirmatory visit 2 to 3 weeks after |
| | neurologic deterioration. Visits are to continue until the last patient's |
| | scheduled Month 6 follow-up visit. |
| Assessments | Screening: History, physical, Karnofsky Performance Status (KPS) |
| | evaluation, magnetic resonance imaging (MRI) of the head, |
| | neurologic examination and symptom assessment, neurocognitive |
| | testing, laboratory evaluations, and serum pregnancy test for women |
| | of childbearing potential. |
| | Treatment visits: Vital signs (pre- and post-MGd treatment on days |
| | of MGd treatment) and adverse events. |
| | Follow-up visits: Neurologic examination, neurologic symptom |
| | assessment, radiologic assessment (per standard of care every 3 |
| | months), neurocognitive testing, concomitant cancer treatments and |
| | corticosteroids. Laboratory evaluation at Month 2 only. Delayed |
| | radiation toxicities through Month 4. For patients who miss |
| | appointments, neurologic status is obtained from outside sources. |
| Inclusion Criteria | A patient meeting the following criteria are enrolled into the study: |
| | Age ≧18 years |
| | Karnofsky performance status ≧70 |
| | Histologically confirmed malignancy with the presence of 1 to 4 |
| | intraparenchymal brain metastases. |
| | Diagnostic gadolinium contrast-enhanced MRI demonstrating |
| | the presence of 1 to 4 unresected brain metastases performed |
| | within 30 days prior to enrollment. |
| | The contrast-enhancing tumor is well circumscribed and has a |
| | maximum diameter ≦4.0 cm in any direction on the enhanced |
| | scan. If multiple lesions are present and 1 lesion is at the |
| | maximum diameter, the other(s) should not exceed 3.0 cm in |
| | maximum diameter. |
| | Each patient must sign a study-specific Informed Consent form |
| Exclusion Criteria | A patient meeting any of the following criteria are excluded from |
| | this study: |
| | Previous cranial radiation, except for patients currently |
| | undergoing WBRT at the protocol specified regimen and who |
| | have not yet received more than 5 doses. |
| | Complete resection of all known brain metastases. Patients who |
| | have undergone subtotal resection are eligible providing residual |
| | disease is ≦4.0 cm in maximum diameter (patients who have |
| | undergone a total resection but have 1-4 recurrent brain |
| | metastases meeting the size criteria are eligible) |
| | Patients with leptomeningeal metastases documented by MRI or |
| | cerebral spinal fluid (CSF) evaluation (lumbar puncture not |
| | required) |
| | Liver metastases (re-staging not required) |
| | Plan to use other experimental therapy during treatment period |
| | (up to completion of SRS) |
| | Clinical or radiologic evidence of progression (other than study |
| | lesion[s]) within one month prior to enrollment. Untreated |
| | disease at other sites will not be considered “progression”. |
| | Patients may receive radiation therapy to other noncranial sites, |
| | as clinically indicated. |
| | Patients with metastases within 10 mm of the optic apparatus so |
| | that some portion of the optic nerve or chiasm are included in |
| | the high dose SRS boost field. |
| | Patients with metastases in the brainstem, midbrain, pons, or |
| | medulla |
| | Plan to use WBRT other than 2.5 Gy × 15 fractions |
| | Planned chemotherapy during WBRT and/or SRS (prior and |
| | subsequent chemotherapy is allowed) |
| | Known history of porphyria |
| | Known history of glucose |
| | Plan to use WBRT other than 2.5 Gy × 15 fractions |
| | Planned chemotherapy during WBRT and/or SRS (prior and |
| | subsequent chemotherapy is allowed) |
| | Known history of porphyria |
| | Known history of glucose-6-phosphate dehydrogenase (G6PD) |
| | deficiency |
| | Known history of HIV infection or AIDS (HIV test not required |
| | for eligibility) |
| | Uncontrolled hypertension (defined as systolic blood pressure >160 mm Hg |
| | and diastolic blood pressure >110 mm Hg on |
| | maximal medical therapy) |
| | Laboratory values as follows: |
| | (a) LDH >1.3 × upper limit of normal (ULN) |
| | (b) ANC <1500/mm3 |
| | (c) Platelets <50,000/mm3 |
| | (d) Creatinine >2.0 mg/dL |
| | (e) Aspartate aminotransferase (AST) or alanine |
| | aminotransferase (ALT) >3 × ULN |
| | (f) Total bilirubin >2 × ULN |
| | Major medical illnesses or psychiatric impairment that in the |
| | Investigator's opinion will prevent administration or completion |
| | of the protocol therapy and/or will interfere with follow-up |
| | Inability to follow study procedures and instructions from study |
| | personnel |
| | Women who are pregnant or lactating (women of childbearing |
| | potential must have a negative serum pregnancy test to enter the |
| | trial. If serum pregnancy test is positive a pregnancy must be |
| | ruled out by ultrasound.) |

Results from the Phase II study outlined above provide information regarding the safety and tolerability of motexafin gadolinium (MGd) with whole brain radiation therapy (WBR'followed by stereotactic radiosurgery (SRS) boost therapy. The study also evaluates the treatmen protocol on lesion size, neurologic progression, neurocognitive progression, and survival. In addition, the study monitors effects of the treatment protocol on radiologic progression and radiologic response. Other parameters evaluated in this trial include determination of whether the MRI signal after 11 doses of MGd improves SRS treatment-planning by identifying and better defining the volume of lesions that can be treated with the SRS boost, when compared to the information obtained using standard contrast agents.

### Example 2

### Therapeutic Regimen of Phase II Trial

Patients meeting the eligibility criteria are treated daily with MGd, 5 mg/kg/day for 10 days during Weeks 2 and 3 of a 3-week course of WBRT (5 days/per week; 37.5 Gy in 15 fractions). MGd is given 2 to 5 hours before each WBRT treatment. One to 14 days (Week 4 or 5within completion of treatment with MGd and WBRT, a single dose of MGd 5 mg/kg is followed by a treatment-planning MRI and SRS boost. Patients optionally receive up to two additional doses of MGd (5 mg/kg) prior to SRS. When the treatment-planning MRI is obtained prior to the day of frame placement and SRS and the last dose of MGd with WBRT (typically thel O dose) was more than 4 days earlier, the MGd is administered prior to the treatment-planning MRI as well as prior to SRS. The treatment schedule is provided below in Table 2.

TABLE 2
Schedule of Therapeutic Regimen

| | |
|---|---|
| Week 1 | Patients receive 2.5 Gy WBRT treatments 1 through 5, typically during |
| | the first 5 days or the study (or prior to enrollment as appropriate). |
| Weeks 2 and 3 | Patients receive infusion of MGd 5 mg/kg over approximately 10 to 30 |
| | minutes, once a day for 10 days, followed by 2.5 Gy of WBRT 2 to 5 |
| | hours later. |
| Week 4 or 5 | Patients receive infusion of MGd 5 mg/kg over approximately 10 to 30 |
| | minutes, 2 hours or more before administration of SRS. The frame for |
| | positioning SRS boost treatment may be placed before or after MGd |
| | infusion. The pre SRS-MRI (with and without standard gadolinium |
| | contrast) to target the SRS boost occurs after the MGd infusion and frame |
| | placement. Next, the SRS boost is administered within 5 hours after MGd |
| | infusion. |

A. Administration of MGd

MGd is supplied in 5% mannitol as a 2.5 mg/mL sterile, preservative-free, dark gr solution, in a single-use 50 mL vial for injection. Prior to use, the MGd is filtered with a pore size ranging from 0.45 gm to 5.0 pm. The filtered solution is transferred into a plastic or glass sterile bag. The MGd solution is administered through a peripheral IV site or a peripherally inserted cen catheter or other indwelling catheter.

Patients receive an infusion of MGd 5 mg/kg over approximately 10 to 30 minutes once a day 2 to 5 hours before each WBRT fraction for 10 days during Weeks 2 and 3 of WBRT treatment. Subsequently, MGd 5 mg/kg is administered once during Week 4 (or 5) 2 hours before administration of SRS boost. Patients may receive one additional dose of MGd 5 mg/kg, if the treatment-planning MRI is obtained more than 4 days following the last dose of MGd and at least day prior to the SRS treatment day.

Antiemetic prophylaxis prior to MGd is recommended but not required during WBRT. Prior to undergoing SRS, patients are strongly urged to intake antiemetics before MGd administration. On the day of SRS treatment, pretreatment with antiemetics prior to MGd dosing minimizes vomiting during the SRS procedures. Patients receive one of the following or equivalent antiemetics before MGd administration at these or other commonly used doses: Z ofran 24 mg orally (PO) 30 minutes before MGd; Z ofran 32 mg, IV; Kytril 2 mg PO 1 hour before MGd; or Kytril 10 μg/kg IV.

Patients may receive anticonvulsants of choice, as clinically indicated. Patients may also receive corticosteroids as clinically indicated. Patients with symptoms, neurologic signs, or imaging evidence of edema or mass effect may receive dexamethasone or other corticosteroids at the time of brain metastasis diagnosis. The recommended starting dose is 8 mg/day of dexamethasone in four divided doses, or equivalent doses of other types of glucocorticoids. If medically necessary the dose may be increased. Patients may continue receiving steroids without tapering the dosage until WBRT is completed. If medically necessary, patients may receive other supportive cancer therapies, such as bisphosphonates, etc.

B. Whole Brain Radiation therapy (WBRT)

WBRT is administered within 1 week of enrollment, using opposed lateral fields, with appropriate shielding. One treatment of 2.5 Gy is given daily 5 days a week, for 3 weeks, for total of 37.5 Gy. Both cranial portals are treated during each treatment session.

Patients may receive additional concomitant radiation therapy other than the pre-specified WBRT and SRS during the course of treatment and thereafter.

C. Stereotactic radiosurgery (SRS)

SRS is delivered within 14 days of completing WBRT. The specific dose of SRS dependent on the size of the lesion being treated as determined by the Investigator. See, for example, Kesserling et al., Int. J. Radiat. Oncol Bio. Phys., 42 (1, Supplement): 263, abstract 207(1998)). The SRS dose used in this study does not exceed the parameters outlined below in Table A radiosurgery dose reduction of up to 25% is permitted at the discretion of the treating physician whenever it is deemed unsafe to deliver the recommended doses either due to the proximity of lesions to each other or to critical normal tissues. SRS dose reduction for multiple brain metastas as described above is considered if the edge of any two lesions are ≦2 cm from each other, if the brainstem dose exceeds 15 Gy, or if the chiasemal or optic nerve dose exceeds 8 Gy.

TABLE 3
Recommended SRS Doses for Varying Lesion Sizes

| Maximum Lesion Diameter | | SRS Dose |
|---|---|---|
| ≦2.0 | cm | 21 Gy |
| 2.1-3.0 | cm | 18 Gy |
| 3.1-4.0 | cm | 15 Gy |

Target volume and isocenter determination is based on a post-whole brain radiation therapy contrast-enhanced MRI scan. The imaging study used to deliver SRS treatment is the same as used to determine size of the metastatic lesion(s). If the treatment-planning MRI is done on the same day as SRS with headframe in place, then MGd dosing precedes the MRI, and both enhanced and unenhanced sequences are obtained. If the treatment-planning MRI is done the day before SRS, MGd is administered if more than 4 days have elapsed since the last dose of MGd, and both enhanced and unenhanced sequences are obtained. For both situations, the final dose of MGd is administered 2 to 5 hours before the SRS treatment.

The dose is prescribed to the isodose surface (50-90% [maximum =100%]), which encompasses the margin of the metastasis, as defined by the treatment-planning MRI. The prescription dose is delivered to the 50 to 90%/o (maximum =100%) isodose surface and is definecd as the minimum dose to the TV. The minimum dose is established by an examination of the dose distribution on each axial level on which the TV has been defined, and/or by the target dose-volumen histogram. Margin expansions are not performed in the treatment planning.

If a patient exhibits one lesion that is >3.0 cm in diameter, each remaining metastas does not exceed 3.0 cm in diameter to remain eligible for the study. This stipulation in size minimizes toxicity in patients having larger volume multiple metastases. In addition, the protocol includes patients having more than 4 lesions for study entry, as may be revealed during the treatment-planning MRI. Patients with 7 or more lesions who receive MGd and SRS are followed through Month 4 and then discontinued from the study.

The patient is treated in the supine position. Adequate immobilization and reproducibility of position is attained. The target volume (TV) covers the brain, and meninges to the foramen magnum. Treatment is delivered through parallel opposed fields that cover the entire cranial contents. Treatment is delivered using megavoltage machines with photon beams ranging from 4 to 8 MV. The minimum dose rate at the midplane in the brain on the central axis is 0.50 Gy/minute. Electron, particle, or implant therapy is not employed.

Stereotactic MRI slice thickness does not exceed 5 mm. The target volume includes the enhancing portion of the metastatic lesion. Surrounding areas of edema are not considered part of the target volume.

If necessary, SRS treatment is re-evaluated at the time of radiation administration in the following scenarios listed below.
(1) An identified metastasis at the time of enrollment exceeds a given upper limit diameter (e.g., 4.0 cm for a solitary metastasis, or 3.0 cm for multiple metastases). If any one metastasis exceeds the limit at the time of radiosurgery, the patient is taken off the study protocol.
(2) If a lesion disappears after the course of conventional radiation, this lesion is not targeted for radiosurgery. If all lesions in a patient disappear, this patient will be taken off the study protocol.
(3) If more than three lesions exist at the time of radiosurgery, radiosurgery is delivererd to up to six treatable lesions that meet the eligibility criteria. Patients with more than 6 lesions, or additional lesions that do not meet the eligibility criteria, do not receive SRS and their participation in the study protocol is discontinued.

### Example 3

### Evaluated Parameters ofPhase II Trial

Patients are followed until termination from the study, death, or the last patient enrolled in the study has completed 6 months of follow-up, whichever occurs first. Evaluations using neurologic exam and symptoms, neurocognitive function, and radiologic scans are performed at monthly visits through Month 9, and then every 3 months until 6 months after the 45^{th} evaluable patient has enrolled. Patients who complete treatment return for a follow-up visit at the Month-2 visit (60±7 days, after start of WBRT). Patients are followed by MRI for radiologic progression every 3 months. A visit window of ±7 days is allowed for each follow-up visit through Month 4. From Month 5 on ward a visit window of ±14 days is allowed. Patients with evidence of neurologic, neurocognitive, or radiologic progression at follow-up return for a confirmatory visit 2 to 3 weeks later. Patients are monitored for radiation toxicity for 3 months following the SRS boost (Month 4 visit), and general safety throughout the treatment period and through the first follow-up visit (Month-2 visit).

A. Neurologic History and Examination

At screening, all patients undergo a history and abbreviated neurologic exam to eli the presenting signs and symptoms of their brain metastases and the presence or absence of major signs and symptoms related to brain metastases. Neurologic assessments are performed within 14 days of the last scheduled dose of WBRT, and at all follow-up visits. A neurologic examination is carried out to examine alertness, orientation, language, speech, cranial nerves, motor strength, sensory deficits, and cerebellar function. Grading scales are provided for each of the neurologic examination components.

If clinical neurologic deterioration is noted at a visit, the patient is scheduled for a confirmatory visit 2 to 3 weeks later, which includes neurologic examination, neurologic symptom collection, KPS, weight, and neurocognitive testing. Neurologic deterioration can include new or worsening aphasia, visual field defect, ataxia., stupor, coma, paresis, numbness, cranial nerve deficits, papilledema, dysarthria, confusion, or seizures.

If a patient is seen by a physician or study personnel for a confirmatory visit or at an off-schedule time point, assessments performed at that time are reported on an unscheduled visit CRF. If a patient misses a visit, the neurologic status is obtained from available outside sources, such as physician's notes, hospital records, and/or conversations with the patient or caregiver(s).

B. Time to Neurologic Progression

Neurologic progression is determined by a qualified central reviewer for each patient after the patient terminates from the study treatment. Each patient is analyzed for the presence of major criteria and minor criteria set forth in Table 4 below.

Major criteria are findings that by themselves are specific and sufficient indicators worsening brain tumors, without further confirmation. Minor criteria are findings that singly are not sufficient, but when in combination with other findings are specific for worsening brain tumors. That is, minor criteria comprise a set of findings consistent with the worsening of a brain tumor, being each finding by itself is not sufficient to declare neurologic progression. In order for a patient to be considered as exhibiting neurologic progression, s/he exhibits an initial combination of 3 minor criteria, followed by confirmation on a subsequent visit.

TABLE 4
Criteria for evaluating neurologic progression

| | |
|---|---|
| Change in Level of | Patients who become stuporous or comatose during the course of |
| Consciousness | study follow-up will be scored as having a neurologic progression |
| | at the time of the event if evidence of other systemic causes has |
| | been effectively ruled out. Data, including laboratory evaluations, |
| | will be available to rule out drug-induced changes, decline in |
| | pulmonary function, metabolic changes in electrolytes or glucose, |
| | overwhelming infection, and hepatic or renal failure. |
| Aphasia/Dysphasia | Patients found to have new onset aphasia or dysphasia will be |
| | scored as having a neurologic progression. |
| Motor Strength | Patients found on physical examination to have a greater than or |
| | equal to 3-grade change from baseline in motor strength in a limb |
| | will be scored as having a neurologic progression. Global motor |
| | strength will be evaluated in all limbs proximally and distally to |
| | ensure that the weakness is not attributable to corticosteroid |
| | myopathy. Investigators will rate patient motor strength using |
| | standard physical exam criteria: |
| | 5 = Active movement against full resistance without evidence of |
| | fatigue (Normal muscle strength) |
| | 4 = Active movement against gravity and some resistance |
| | 3 = Active movement against gravity |
| | 2 = Active movement of the body part with gravity eliminated |
| | 1 = A barely detectable flicker or trace of contraction |
| | 0 = No muscular contraction detected |
| New Visual Field | Patients developing a new visual field defect will be scored as |
| Defect | having a neurologic progression. |
| Ataxia | Patients developing a 2 grades or greater worsening from baseline |
| | in their gait will be scored as having a neurologic progression. Gait |
| | abnormalities cannot result solely from lower extremity motor |
| | weakness. Ataxia will be scored according to the following scale: |
| | 0 = No evidence of ataxia |
| | 1 = Unsteady but able to walk |
| | 2 = Able to walk with unilateral assistance |
| | 3 = Able to walk with bilateral assistance |
| | 4 = Requires wheelchair, unable to walk |
| Executive | Executive function is felt to be the most specific indicator of |
| Function | neurologic deterioration found on neurocognitive testing that can |
| | result from the presence of a brain tumor. Patients developing a 2 |
| | point or greater deterioration from baseline in their z-scores on the |
| | Trailmaking Test B and COWA Test will be scored as having |
| | neurologic progression. Both of the deteriorations in these tests |
| | must be confirmed on consecutive study visits unless both |
| | deteriorations are found on the last visit before death, study |
| | termination or before additional brain directed therapy. |

Minor Criteria

| | |
|---|---|
| Change in | A change in orientation from baseline affecting 2 of 3 areas |
| Orientation | (person, place or time) would be considered a minor criterion. In |
| | order to be oriented to person, the patient must be able to identify |
| | him/herself. In order to be oriented to place, the patient must be |
| | able to identify the location (eg hospital). In order to be oriented to |
| | time, the patient must be able to identify the month and year. |
| Change in Motor | A 2-grade change from baseline in motor strength in a limb will be |
| Strength of 2 | considered a minor criterion. The motor strength scale is presented |
| Grades | above. The motor strength of all limbs will be evaluated to ensure |
| | that the weakness is not attributable to corticosteroid myopathy |
| Loss of Sensation | Loss of light touch sensation in a limb will be considered a minor |
| in a Limb | criterion. At study visits, investigators will assess patients for loss |
| | of light touch in the limbs. |
| Facial Weakness | The development of a new facial weakness will be considered a |
| | minor criterion. |
| Facial Numbness | The development of new facial numbness will be considered a |
| | minor criterion |
| New Onset | The development of new seizures during the study follow-up |
| Seizures | period will be considered a minor criterion. If the patient has a |
| | prior history of a seizure, worsening of the seizure type or |
| | increasing frequency would not meet the criterion. After a |
| | diagnosis of seizure activity is established, further new diagnoses |
| | of the same seizure activity cannot be repeated; this criterion will |
| | not require confirmation and will be considered a minor criterion |
| | from that point forward. However, when other data are available, |
| | other causes of seizures will be ruled out to establish a brain |
| | metastases etiology. |
| Neurocognitive | A worsening of a z-score by 2 points in the HVLT's recall or |
| Tests | delayed recall, the Trailmaking Test B, or the COWA test will be |
| | considered a minor criterion. If multiple tests within the same |
| | functional domain worsen, they will only count as one minor |
| | criterion (eg, worsening in the Hopkins verbal learning tests recall |
| | and delayed recall on the same visit are considered as 1 minor |
| | criterion). HVLT (recognition) and Trailmaking Test A, because |
| | of their low sensitivity, will not count towards the minor criteria. |
| Pupils | Changes resulting in unequal or unreactive pupils during study |
| | follow-up will be considered a minor criterion. |
| Gait Abnormality | The development of a 1 grade worsening in a gait abnormality |
| (Ataxia) | constitutes a minor criterion. Gait abnormalities are graded |
| | according to the scale set above. Investigators will assess gait by |
| | asking patients to walk, if possible, unassisted. |
| Finger-to-Nose-to- | New abnormalities detected upon assessment of cerebellar |
| Finger Exam | function using the finger-to-nose-to-finger test will be considered a |
| | minor criterion in the absence of motor weakness in the upper |
| | extremity used during this examination. |
| Oculomotor Palsy | The development of an oculomotor palsy because of deficits in |
| | cranial nerves III, IV or VI will be considered a minor criterion. |
| | To assess for oculomotor palsy investigators will be asked to |
| | assess patients for deficits in extraocular motion or symptoms of |
| | double vision that resolve when one eye is covered. |
| Papilledema | Upon fundoscopic examination, the detection of new papilledema |
| | will be considered a minor criterion. |
| Dysarthria | The development of new dysarthria will be considered a minor |
| | criterion. |

C. Neurocognitive Tests

Neurocognitive testing is performed at screening before enrollment, within 14 day the last dose of WBRT, and at each follow-up visit. The neurocognitive tests, with references and approximate administration times, are listed in Table 5 and described generally below.

TABLE 5
Neurocognitive and Functional Status Test Battery

| | | Approximate |
|---|---|---|
| | | Administration |
| Domain | Test | Time |
| Memory | Hopkins Verbal Learning Test | 5 min |
| Executive function | Controlled Oral Word Association | 5 min |
| Visual motor speed | Trail Making Test Part A | 5 min |
| Executive function | Trail Making Test Part B | 5 min |

Hopkins Verbal Learning Test (HVLT)

The HVLT consists of six unique forms, each with a 12-item word list composed of four words from each of three semantic categories. The HVLT evaluates patients' ability to memorize and recall. See Brandt J., “The Hopkins Verbal Learning Test: development of a new memory test with six equivalent forms,” Clin. Neuropsychol., 5:125-142 (1991), the contents of which are hereby incorporated by reference.

Trail Making A and B

The Trail Making test consists of two parts in which the patient connects numbered circles in a numeric sequence (Part A) and in a numeric plus alphabetical sequence (Part B). Part evaluates visual-motor scanning speed, and Part B evaluates executive function. See Benton et al., Controlled word association. In: Multilingual Aphasia Examination Manual of Instructions. 3rd Edition. Iowa City, Iowa, AJA Associates Inc. (2000), the contents of which are hereby incorporated by reference.

Controlled Oral Word Association (CO WA)

The COWA test is an oral fluency examination in which the patient is required to make verbal associations to different letters of the alphabet by saying all the words he or she can think of that begin with a given letter. Three letters of progressively increasing associative difficulty are presented successively as stimuli. See Lezak et al., Neuropsychological Assessment, Third Edition, New York, N.Y., Oxford University Press (1995).

D. Radiological Assessment

Diagnostic brain MRI data is provided in the form of duplicate films and/or digital data and hard-copy imaging reports. If the diagnostic scan was performed more than 30 days before enrollment, contrast-enhanced MRI (±Gd) is repeated during the screening period. All available scans are reviewed by the Investigator or designee before enrollment to rule out leptomeningeal or subarachnoid spread of tumor.

A treatment-planning MRI (with and without standard gadolinium contrast) is performed prior to SRS. Information from the treatment-planning MRI is used to compare changes in lesion size and number from the pretreatment (or diagnostic) MRI and to evaluate radiological progression. The treatment-planning MRI may occur with the sterotactic frame in place following administration of MGd on the SRS treatment day. If the MRI is performed prior to the SRS treatment day, MGd is administered on that day prior to the MRI if more than 4 days have elapsed since the last dose of MGd. Regardless of when the treatment-planning MRI is performed, the MGd is administered 2 to 5 hours prior to SRS.

Radiological response is evaluated at month three. Radiological progression is assessed at month three and then every three months with the MRI scan obtained during routine clinical follow-up. Radiological progression is defined as the occurrence of new brain metastases the development of leptomeningeal involvement, the reappearance of old lesions, or an increase in the sum of lesion sizes by 25%. The lesion size is defined as the product of the longest perpendicular diameters. Radiological progression is documented on a CRF. Radiological response is defined as a decrease in the lesions size of all lesions by at least 50%, without the occurrence of new lesions. Complete response is defined as the complete resolution of all brain metastases without the occurrence of new lesions.

### Example 4

### Clinical Endpoints ofPhase II Trial

A. Primary Endpoints

The primary endpoint of the study is determined by the proportion of patients that have a minimum of one neurologic toxicity within three months of SRS boost. The observed proportion of patients with neurologic toxicities and the corresponding exact binomial 95% confidence interval with mid-p adjustment is calculated.

B. Secondary Endpoints

The secondary endpoints of the study are evaluated by the following factors: change in lesion size and number between screening MRI and SRS treatment-planning MRI; time to neurologic progression or death with evidence of neurologic progression; time to neurocognitive progression; and time to all-cause mortality.

Change in Lesion Size and Number between Screeninz MRI and SRS Treatment-planning MRI

Change in lesion counts at screening and pre-SRS is summarized by frequency, mean, standard deviation, median, minimum, and maximum. Changes in count from baseline, number of new tumors visible at pre-SRS scan, and numttber of baseline tumors not visible at pre-SRS scan are also summarized as frequency, mean, standard deviation, median, minimum, maximum. Lesion sizes are calculated as the product of the longest perpendicular cross sectional diameters on the slice in which each lesion appears the largest on the T-1 weighted contrast-enhanced images. Patient means and patient totals are summarized as mean, standard deviation, median, minimum, and maximum for the screening and pre-SRS scan. Average and total change are calculated for each patient and summarized across all patients.

Time to Neurologic Progression or Death with Evidence of Neurologic Progression

Patients who are still alive and have no evidence of neurologic progression at the t of termination are censored at the time they terminate the study. Patients who die without evidence of neurologic progression are censored on the date of their death. Estimates of time to neurologic progression or death with evidence of neurologic progression are plotted using the Kaplan-Meier method. A point estimate and 95% confidence interval for the median time to neurologic progression or death with evidence of neurologic progression is presented.

Time to Neurocoznitive Progression

Patients who die or terminate the study without evidence of neurocognitive progression are censored. The raw neurocogrnitive data is transformed to z-scores based on normative data. Three specified deteriorations in z-scores, 1.5, 2, and 3, are used to respectively g three definitions of progression. The progression time is defined as the time of the first drop in z-score with a subsequent confirmation. For each test and each of the three progression definitions, estimates of time to neurocognitive progression are plotted using the Kaplan-Meier method. A po estimate and 95% confidence interval for the median time to neurocognitive progression is presented for each case.

Time to All-Cause Mortality

Time to all-cause mortality is defined as the time elapsed from the start of WBRT to death from all causes during the entire study -period (i.e., 6 months after the last patient is enrolled Patients still alive at the end of the study, or at termination, are censored at that time. Estimates of time to all-cause mortality are plotted using the Kaplan-Meier method. A point estimate and 95% confidence interval for the median time to all-cause mortality is presented.

C. Exploratory Endpoints

The exploratory endpoints evaluated in this of the study are time to radiologic progression, and radiologic response rate at month three.

Estimates of time to radiologic progression are plotted using the Kaplan-Meier method. A point estimate and 95% confidence interval for the median time to radiologic progression is presented. Patients who die or terminate the study without evidence of radiologic progression arebe censored.

Using the definition of radiologic response provided above, the observed proportion of patients who have responded at month three are calculated. An exact binomial confidence interval with mid-p adjustment is also presented.

Unless stated otherwise, all publications and patent applications mentioned in this specification are herein incorporated by reference to the same extent as if each individual publicatin or patent application was specifically and individually indicated to be incorporated by reference.

What is claimed is:

1. A method of treating cancer in a patient, comprising:
(a) administering at least 5 mg/kg of a high-purity texaphyrin-metal complex or a pharmaceutically acceptable derivative to the patient; and
(b) providing, within at least 24 h after receiving a dose of a texaphyrin-metal complex, at least 10 Gy of radiation to at least one lesion in the patient.

2. The method of claim 1, wherein the high-purity texaphyrin-metal complexes is Motexafin Gadolinium (MGd).

3. The method of claim 1, wherein the patient is further administered at least one dose of a non-texaphyrin gadolinium complex.

4. The method of claim 3, wherein the NIGd is administered at least 4 hours prior to receiving at least 10 Gy to at least one lesion.

5. The method of claim 4, wherein at least five doses of MGd have been administered in the three weeks prior to receiving at least 10 Gy to at least one lesion.

6. The method of claim 1, further comprising the step of providing hyperfractionated radiation therapy to the patient.

7. The method of claim 1, further comprising the step of providing hyp ofractionated radiation therapy to the patient.

8. The method of claim 2, wherein the at least 10 Gy of radiation is provided by stereotactic radio surgery.

9. The method of claim 6, wherein the high-purity texaphyrin-metal complex or a pharmaceutically acceptable derivative is administered prior to each time the patient receiving hyperfractionated radiation therapy.

10. The method of claim 9, wherein the patient receives MGd during the second and third week of treatment of hyperfractionated radiation therapy.

11. The method of claim 6, wherein the patient receives a total of about 30 to about 50 Gy of radiation during hyperfractionated radiation therapy.

12. The method of claim 1, wherein the patient receives at least about 15 Gy of radiation to at least one lesion.

13. The method of claim 9 wherein about ten doses of MGd in an amount of up to about 5 mg/kg is administered daily during the second and third week of treatment of hyperfractionated radiation therapy.

14. The method of claim 1, wherein the size of at least one lesion is reduced by at least about 50%.

15. The method of claim 1, wherein the patient exhibits about 1 to 4 brain metastases from a solid tumor.

16. An improved method for treating cancer in a patient undergoing radiation therapy, wherein the improvement is administering an effective amount of a high-purity texaphyrin-metal complex or a pharmaceutically acceptable derivative before the patient receives stereotactic radio surgery.

17. The method of claim 16, wherein the radiation therapy further comprises whole-brain radiation therapy (WBRT).

18. The method of claim 16, wherein the high-purity texaphyrin-metal complex is MGd.

19. The method of claim 16, wherein the patient is further administered at least one dose of a non-texaphyrin gadolinium complex.

20. The method of claim 17, wherein the cancer is brain metastases and the patient exhibits 1 to 4 brain metastases from a solid tumor.

21. The method of claim 20, wherein the brain metastases' size is reduced by at least about 50%.

22. A method of treating metastases in a patient, comprising:
(a) administering an effective amount of motexafin gadolinium (MGd) to the patient; and
(b) performing stereotactic radiosurgery on the area of the patient's body comprising the metastases.

23. The method of claim 22, wherein the MGd is administered prior to performing the stereotactic radiosurgery on the patient.

24. The method of claim 23, further comprising the step of performing WBRT on the patient.

25. The method of claim 24, wherein the MGd is further administered prior to the patient undergoing WBRT.

26. The method of claim 25, wherein the patient exhibits 1 to 4 metastases from a solid tumor.

27. The method of claim 26, wherein the brain metastases' size is reduced by at least about 50%.
